(19) United States
(12) Patent Application Publication
(10) Pub. No.: US 2022/0378597 A1
(43) Pub. Date: Dec. 1, 2022

(54) Universal Ankle-Foot Orthoses

(71) Applicant: Shireen Thor, Coronado, CA (US)

(72) Inventor: Shireen Thor, Coronado, CA (US)

(21) Appl. No.: 17/804,181

(22) Filed: May 26, 2022

Related U.S. Application Data
(60) Provisional application No. 63/193,067, filed on May 26, 2021.

(51) Int. Cl. A61F 5/01

(52) U.S. Cl. CPC A61F 5/0111

(57) ABSTRACT

The present invention is ankle-foot orthoses and related accessories. In one variation, an AFO comprises a bottom dual strut arm, a top strut arm, a foot plate, foot plate slots, and a support strap. In another variation, an AFO comprises a D-ring. In yet another variation, an AFO comprises an angular adjustment gear mechanism. In yet another variation, an AFO comprises spring elements and an adjustment mechanism. In yet another variation, an AFO comprises a foot plate and toeplate with teeth or a length-adjuster that allows for length adjustment. In yet another variation, an AFO is integrated into a sock and includes tensioning elements. In one foot plate variation, a foot plate comprises electrical components that provide gait analysis functionality. In one inner boot variation, an inner boot and support straps are combined into an integrated hammock system. In another variation, the inner boot comprises slots.

### PRIORITY

This application claims the benefit of priority to U.S. Provisional Patent Application No. 63/193,067 entitled “Foot Ankle Orthoses Apparatus and Method of Manufacture and Use” filed May 26, 2021, the contents of which are incorporated herein by reference in its entirety.

### RELATED APPLICATIONS

This application is generally related to the subject matter of co-owned U.S. patent application Ser. No. 15/977,880 entitled “METHODS AND APPARATUS FOR HUMAN ANATOMICAL ORTHOSES” filed May 11, 2018, the foregoing incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### TECHNICAL FIELD

The present disclosure relates generally to the field of orthotics. Specifically, the present disclosure is directed to orthotics that correct disorders of the lower limbs.

### DESCRIPTION OF RELATED TECHNOLOGY

An ankle-foot orthosis (plural “orthoses”), or AFO, is a support intended to control the position and motion of the ankle, to compensate for weakness, or to correct deformities. AFOs can be used to support weak limbs, or to position a limb with contracted muscles into a more normal position. In addition, AFOs are used to control foot drop caused by a variety of neurologic and musculoskeletal disorders. Due to the common use for addressing foot drop, AFO has become synonymous with the term “foot-drop brace”.

The goal of AFO use is to stabilize the foot and ankle and provide toe clearance during the swing phase of gait. This helps decrease the risk of catching the toe and falling.

A typical AFO creates an L-shaped frame around the foot and ankle, extending from just below the knee to the metatarsal heads of the foot. AFO's can be purchased off the shelf or can be custom molded to an individual wearer, and can be fabricated of a variety of materials, including heat-moldable plastics, metals, leathers, and carbon composites. AFOs are the most used orthoses, making up about 26% of all orthoses provided in the United States.

Drop foot is a common medical condition that can be caused by many different pathological conditions. These conditions may be caused by trauma in which the peroneal nerve that innervates the peroneal muscles becomes damaged. Drop foot may also be present following a stroke, or because of various disorders such as multiple sclerosis (MS) and amyotrophic lateral sclerosis (ALS), or after injury. Many orthotic treatments exist for the treatment of drop foot including, for example: rigid ankle-foot orthoses (AFOs); semi-rigid foot orthoses; soft AFOs (such as “Foot Up”-type devices or a soft ankle brace with straps); and functional electrical stimulation systems. For example, carbon fiber AFOs are known for their lightweight and low-profile nature making them easier to fit into a shoe as compared with non-carbon fiber AFOs that are manufactured from fabric and/or plastic.

An estimated 20% of stroke survivors suffer from spastic drop foot (the inability to dorsiflex the foot) which can lead to insufficient toe clearance during the swing phase of gait. Consequently, ankle-foot orthoses (AFOs) are commonly prescribed to address the resulting body segment alignment and ankle joint motion. However, inconsistent alterations to spatial, temporal, and sagittal plane kinematic gait variables have hindered understanding of the biomechanical foundation underlying these benefits.

Existing AFO devices are designed to address deficiencies (such as drop foot) by controlling dorsiflexion or plantar flexion of a wearer's foot. However, many patients also have additional underlying conditions such as, for example, valgus or varus deformities and instability associated with their ankle and/or knees which existing AFO solutions do not address well.

Moreover, various anatomical differences between patients as well as varying degrees of severity for their conditions requires medical practitioners to stock many size and stiffness options for these AFO devices to properly address any given patient's specific medical condition. In addition to these varying sizes, AFO devices may also come in a variety of different stiffnesses such as, for example, light stiffness, standard stiffness, and maximum stiffness.

Accordingly, despite the wide variety of the foregoing solutions, there remains a salient need for an orthotic device that addresses the foregoing problems by: providing varying stiffness support for everyday use dependent upon a given patient's medical condition; is comfortable to wear; is easy to put on and take off by the wearer; and can be easily adjusted throughout the day by either the patient or practitioner in order to provide an appropriate amount of support dependent upon the activity level required of the patient.

### BRIEF DESCRIPTION OF THE DRAWINGS

FIG. 1A illustrates a perspective view of one variation of an AFO.

FIG. 1B illustrates the “donning” or “doffing” of one variation of an AFO.

FIG. 1C illustrates a side view of one variation of an AFO.

FIG. 1D illustrates a perspective view of one variation of an AFO.

FIG. 1E illustrates a perspective view of another variation of an AFO.

FIG. 1F illustrates a perspective view of one variation of angular adjustment gears.

FIG. 1G illustrates a close-up perspective view of one variation of angular adjustment gears.

FIG. 1H illustrates a side view of one variation of an AFO.

FIG. 1I illustrates a side view of one variation of an AFO.

FIG. 1J illustrates an exploded view of one variation of an angular adjustment bumper mechanism.

FIG. 1K illustrates a side view of one variation of an AFO.

FIG. 1L illustrates an exploded view of one variation of an angular adjustment bumper mechanism.

FIG. 1M illustrates a perspective view of several positions of one variation of an angular adjustment bumper mechanism.

FIG. 1N illustrates a close-up side view of one variation of an adjustment mechanism.

FIG. 1O illustrates a close-up side view of another variation of an adjustment mechanism.

FIG. 1P illustrates a close-up side view of another variation of an adjustment mechanism.

FIG. 2A illustrates a perspective view of one variation of an inner boot.

FIG. 2B illustrates a side view of one variation of an inner boot.

FIG. 2C illustrates a side view of another variation of an inner boot.

FIG. 2D illustrates a perspective view of one variation of an AFO.

FIG. 2E illustrates a perspective view of another variation of an AFO.

FIG. 2F illustrates a side view of one variation of an inner boot.

FIG. 2G illustrates a front view of one variation of an inner boot.

FIG. 2H illustrates a perspective view of one variation of an AFO.

FIG. 2I illustrates a perspective view of another variation of an AFO.

FIG. 3 illustrates a top view of one variation of the electrical components of a foot plate.

FIG. 4A illustrates a top view of one variation of an AFO.

FIG. 4B illustrates a top view of another variation of an AFO.

FIG. 5 illustrates a perspective view of one variation of an AFO.

FIG. 6A illustrates a perspective view of another variation of an AFO.

FIG. 6B illustrates a perspective view of another variation of an AFO.

FIG. 6C illustrates a perspective view of another variation of an AFO.

FIG. 7 illustrates a frontal-oblique view of one variation of an AFO.

FIG. 8A illustrates a front view of one variation of an AFO.

FIG. 8B illustrates a close-up front view of one variation of an AFO.

FIG. 9 illustrates a top view of one variation of an AFO.

### DETAILED DESCRIPTION

In the following detailed description, reference is made to the accompanying drawings which form a part hereof wherein like numerals designate like parts throughout, and in which is shown, by way of illustration, embodiments that may be practiced. It is to be understood that other embodiments may be utilized, and structural or logical changes may be made without departing from the scope of the present disclosure. Therefore, the following detailed description is not to be taken in a limiting sense, and the scope of embodiments is defined by the appended claims and their equivalents.

Aspects of the disclosure are disclosed in the accompanying description. Alternate embodiments of the present disclosure and their equivalents may be devised without departing from the spirit or scope of the present disclosure. It should be noted that any discussion herein regarding “one embodiment”, “an embodiment”, “an exemplary embodiment”, and the like indicate that the embodiment described may include a particular feature, structure, or characteristic, and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments may be utilized in connection or combination with those of any other embodiment discussed herein.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

Moreover, while variations described herein are primarily discussed in the context of foot and ankle orthoses for the treatment of drop foot, it will be recognized by those of ordinary skill that the present disclosure is not so limited. In fact, the principles of the present disclosure described herein may be readily applied to other parts of the anatomy of a human, and for treatment of conditions other than drop foot. For example, many common injuries, such as a partial or complete tear of a tendon (e.g., a biceps tendon, a triceps tendon, an Achilles tendon, and the like), may require an individual to rest the injured tendon, whether surgical or non-surgical treatment is required. Accordingly, the principles described herein may be readily adapted for use with other portions of the anatomy. For example, the drop foot sock devices described herein may be readily adapted for use on the arm, elbow, shoulder, knee, where movement, whether in extension or flexion, may need to be constrained to facilitate recovery from, for example, an injury or other medical condition.

Ankle-Foot Orthoses with Universal Strapping System

As a brief aside, human anatomy can be described with reference to three planes: a “coronal” (frontal) plane, a “sagittal” (lateral) plane, and an “axial” (transverse) plane. The coronal plane is a vertical plane that divides the body or any of its parts into anterior and posterior portions. The sagittal plane is a vertical plane that divides the body or any of its parts into right and left portions. The axial plane is a horizontal body that divides the body or any of its parts into upper and lower portions. Additionally, the terms “superior” and “inferior” refer to the relative orientation from the top or head of the body (e.g., the hands are part of the superior extremity, feet are part of the inferior extremity). The terms “medial” and “lateral” refer to relative orientations from the midpoint of the body in the sagittal plane (e.g., the first “big” toe is located on the medial side of the foot, the fifth “pinky” toe is located on the lateral side of the foot). The terms “proximal” and “distal” refer to the relative orientation from the midpoint of the body in the transverse plane (e.g., the proximal end of the femur connects to the hip, the distal end of the femur connects to the knee).

Additionally, as used herein, the term “clinician” refers to a person or entity that provides care to a “patient.” Similarly, “clinical” use refers to usage scenarios that are conducted under the guidance and supervision of a clinician; in contrast, “personal” use refers to unsupervised usage scenarios (e.g., everyday usage). While the following discussions are presented in the context of a human clinician and a human patient, artisans of ordinary skill may readily adapt the techniques to non-human clinicians and/or patients (e.g., machine and/or animal applications). As used herein, the term “user” refers to an entity that uses the orthoses. In some applications, the user may be a clinician fitting an orthoses to a patient or assessing the patient's gait based on the orthotics usage. In other applications, the user may be a patient donning/doffing the orthoses. As discussed in greater detail herein, the universal orthoses provide significant benefits for both clinicians and patients alike.

As used herein, the term “universal” refers to orthotics that provide users with numerous fitting options for a wide variety of conditions and disabilities. Notably, existing ankle-foot orthosis (AFO) which are typically prescribed as fixed one-piece constructions that are made for a specific infirmity e.g., a patient that has either dorsiflexion or plantar flexion issues. Anecdotally, however, patients seldom suffer from just one condition, e.g., a stroke survivor may have a condition that affects both dorsiflexion and plantar flexion. Rather than treating just the most severe symptoms and ignoring less severe impediments, the AFOs described throughout may combine multiple components and/or fitting options that can be interchanged according to the patient's individualized needs.

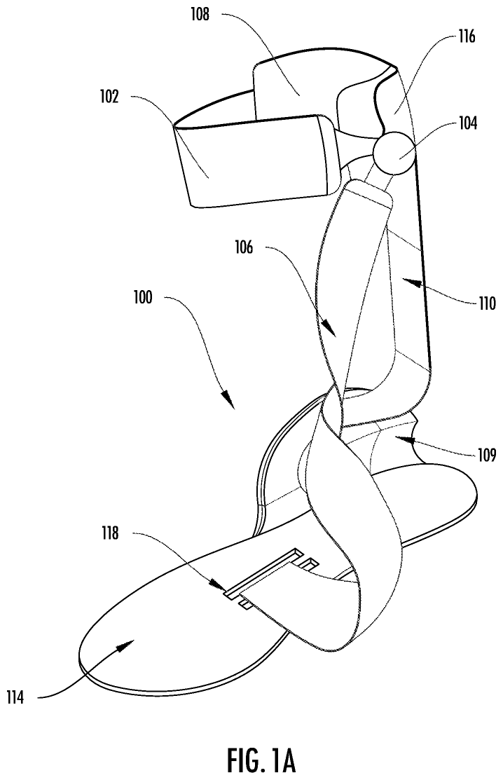

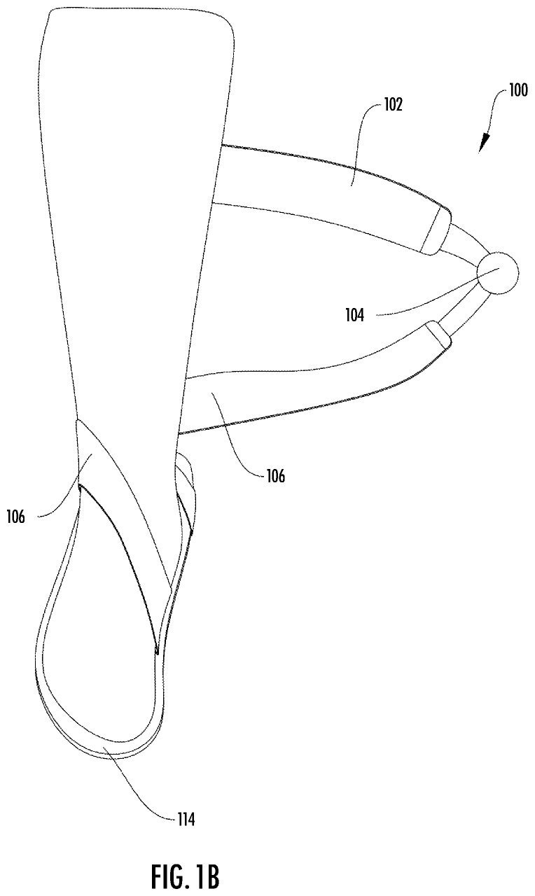

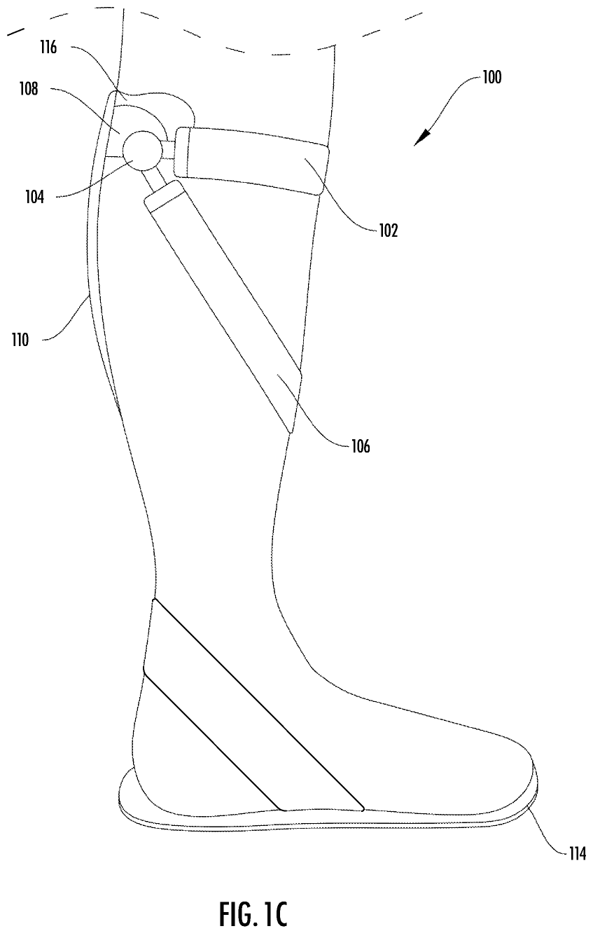

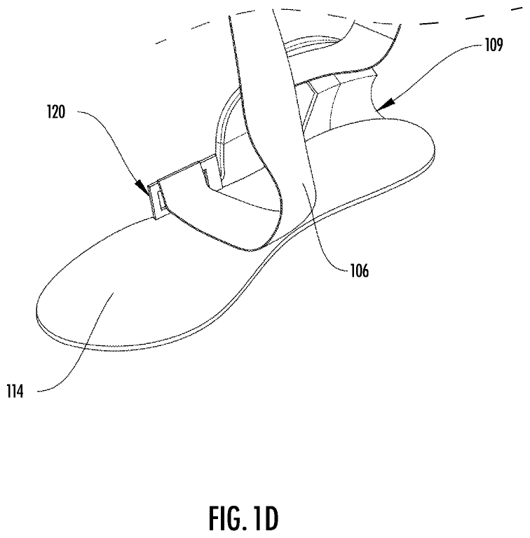

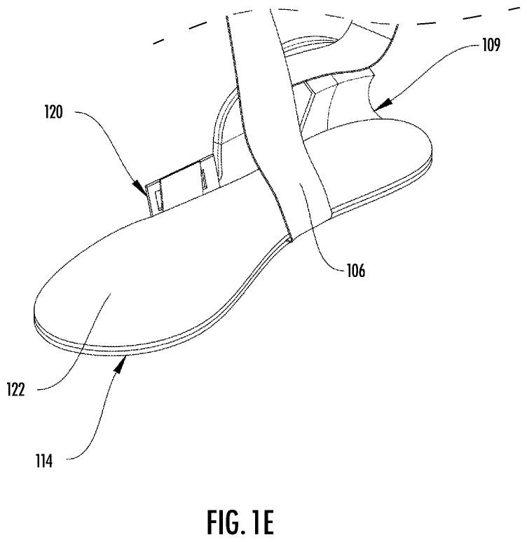

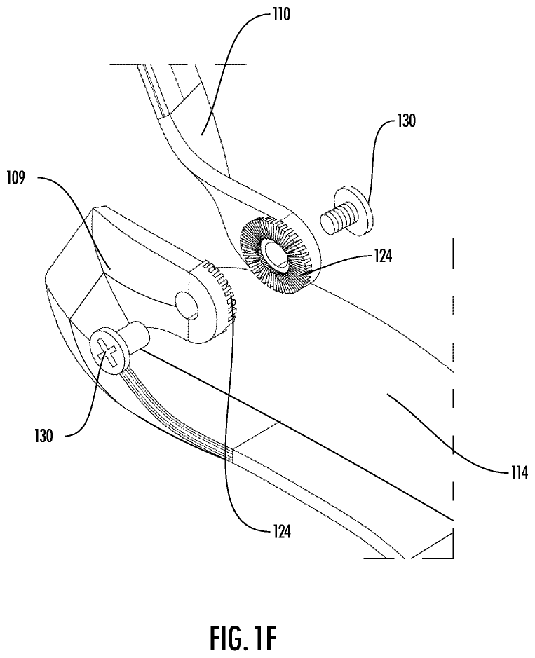

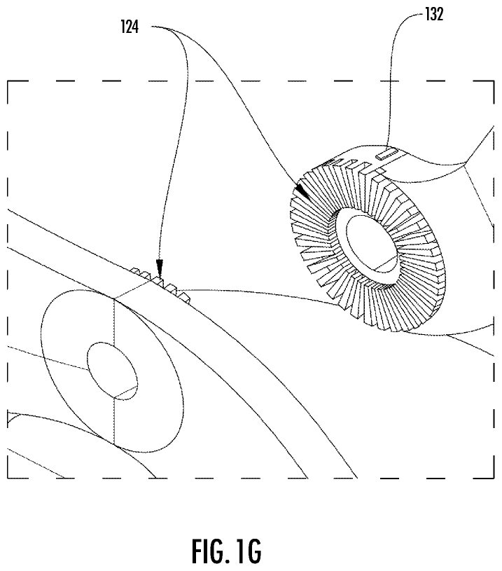

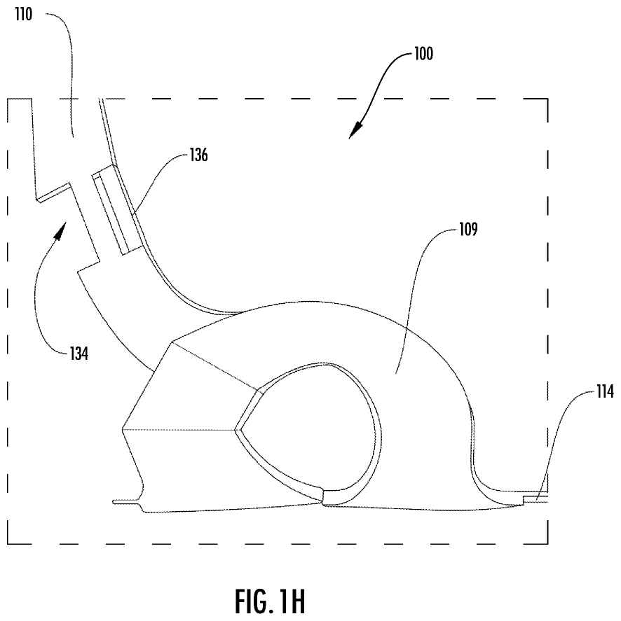

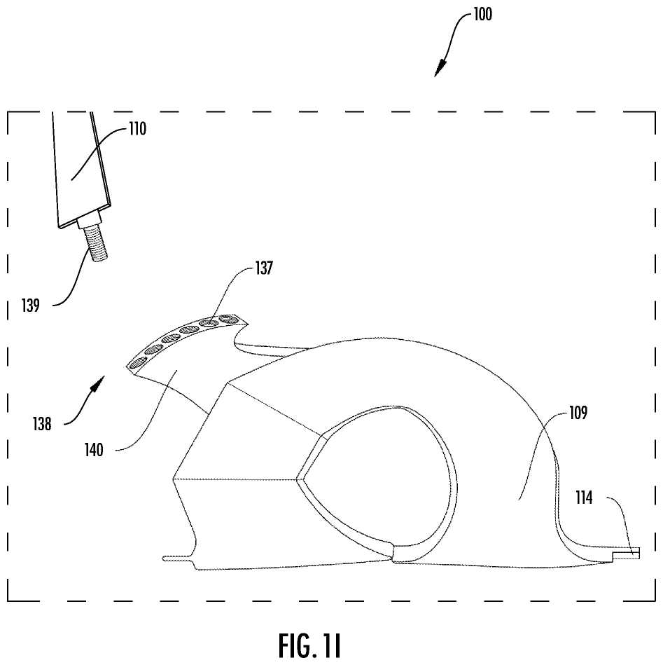

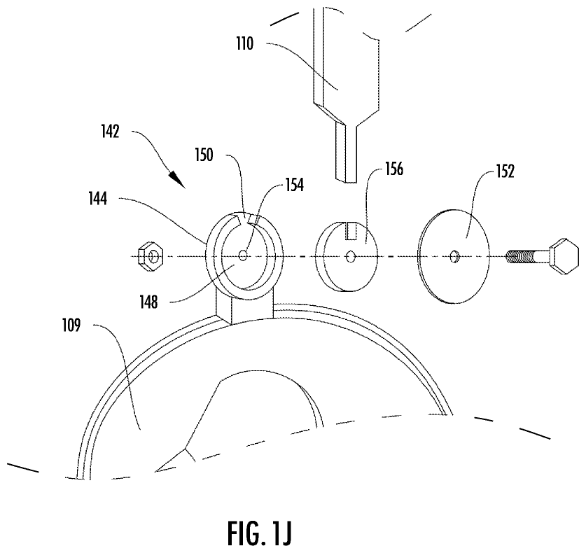

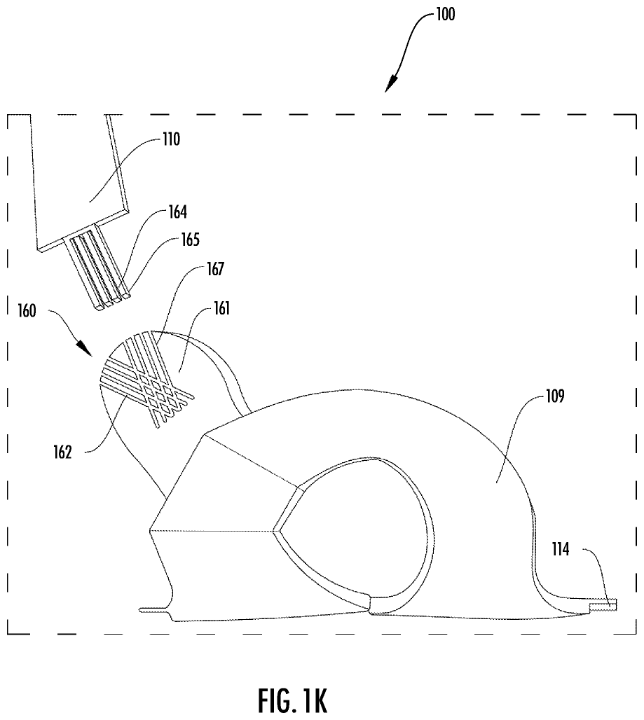

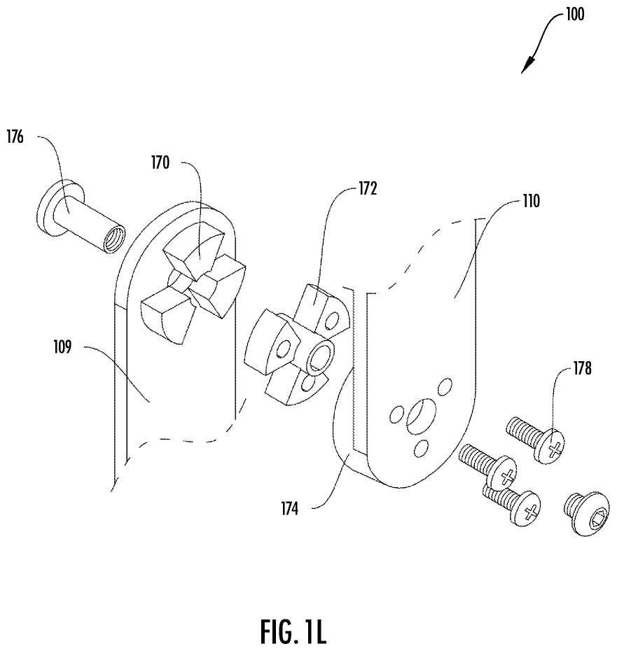

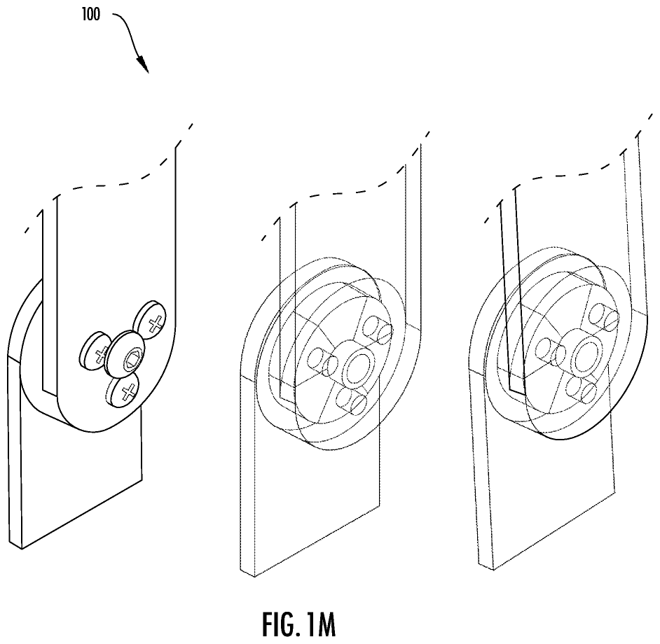

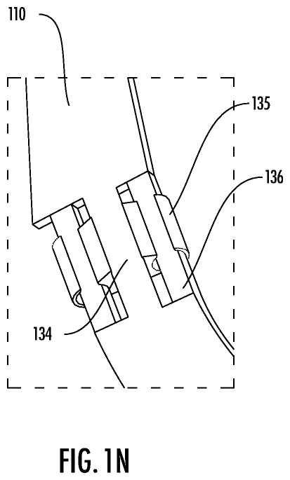

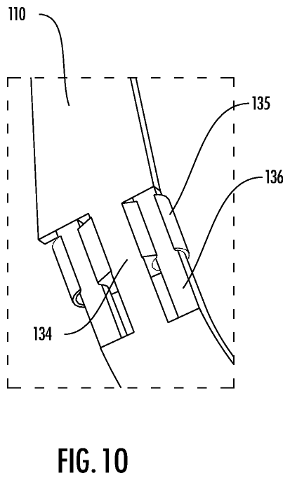

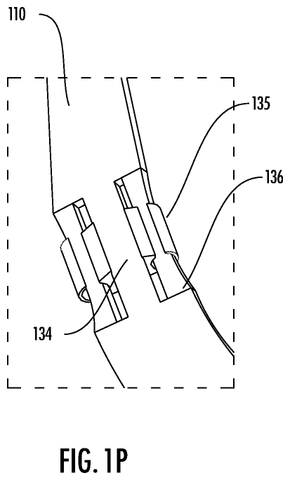

Referring now to FIGS. 1A-1C, one exemplary ankle-foot orthosis (AFO) 100 is shown and described in detail. For example, FIG. 1A illustrates a rigid or semi-rigid AFO that includes a dynamic semi-flexible foot plate 114, a bottom strut dual arm 109, a top strut arm 110, and a supporting structure 108 that is configured to fit adjacent to a patient's knee. The orthosis 100 may further include an adjustable strap 102 which may be secured to anchoring fabric 116 that is disposed on the supporting structure 108. In some variations, the supporting structure 108 may be manufactured from a heat-moldable plastic, metal, leather, a carbon composite material, or various combinations of the foregoing.

The bottom strut dual arm 109 may be positioned laterally on the foot plate 114, ideal for patients with medial ankle instability, that evert, pronate, or have valgus. Alternatively, the bottom strut dual arm 109 may be positioned medially, ideal for patients with lateral ankle instability, that invert, supinate, or have varus. The AFO 100 may have lateral or medial bottom strut dual arm 109 positions, leading to an open-heel design that eliminates pressure sores. The bottom strut dual arm 109 may have a horseshoe shape that allows the AFO 100 to comfortably cradle the patient's medial malleolus or lateral malleolus. In some variations, the bottom strut dual arm 109 may be manufactured from a heat-moldable plastic, metal, leather, a carbon composite material, or various combinations of the foregoing. In another variation, the bottom strut dual arm 109 may only have one arm.

The top strut arm 110 may be configured to be disposed around the foot and ankle either medially, laterally, or posteriorly and run up along a patient's calf muscle. In some variations, the top strut arm may be manufactured from a heat-moldable plastic, metal, leather, a carbon composite material, or various combinations of the foregoing.

A support strap 106 may be anchored on the foot plate slots 118 by having an end section loop around the middle piece of the foot plate slots 118 and reattach to itself. The end section of the support strap 106 may be a permanent feature that is not removable from the foot plate slots 118 or the end section of the support strap 106 may be reconfigurable and removable from the foot plate slots 118 by using, for example, a hook and loop fastener (e.g., Velcro®), a clasp, a button, and/or any other suitable type of fastening mechanisms. This anchoring arrangement may allow multiple unique support strap configurations.

Referring now to FIG. 1B, orthosis 100 is shown being put on (“donning”), or taken off (“doffing”), the leg of a patient. As can now more readily be seen, the support strap 106 may be wrapped in, for example, a clockwise (or counterclockwise) direction around the lower leg of the patient where both the adjustable strap 102 and support strap 106 may be attached to a rotary tensioning system 104. Accordingly, when the rotary tensioning system 104 is secured to, for example, anchoring fabric 116, both the adjustable strap 102 and support strap 106 may be simultaneously placed under tension through adjustments made through the rotary tensioning system 104. Such simultaneous tensioning eases the donning and doffing of the orthosis 100 so that the device may be adjusted using a single hand. Such a configuration may be particularly advantageous for patients that may have, for example, an impairment to one of their hands and/or arms as the orthosis 100 may be put on (or taken off) using one hand. In some implementations, two (or more) rotary tensioning mechanisms 104 may be included with the orthosis 100. For example, one rotary tensioning mechanism 104 may assist with the tensioning of the adjustable strap 102, while another rotary tensioning mechanism 104 may assist with the tensioning of the support strap 106. In some implementations, the support strap 106 may be obviated altogether and hence, a single rotary tensioning mechanism 104 may assist with the tensioning of the adjustable strap 102.

FIG. 1C illustrates orthosis 100 after an appropriate amount of tension has been applied via rotary tensioning system 104. The orthosis 100 as shown in FIGS. 1A-1C may include carbon fiber in, for example, the strut 110 and/or the foot plate 114. An additional dorsiflexion (and/or plantar flexion) support may be provided via inclusion of the support strap 106 (which may be elastic and/or inelastic in portions of the strap 106). In some variants, the direction of the direction of rotation for the support strap 106 may also provide inversion/eversion (valgus/varus) support to ensure proper tibia alignment throughout the gait. In one specific variant, strap 106 includes a rotary tensioning system 104 (e.g., a BOA® dial) that allows for two (or more) straps to be adjusted at the same time; this enables better fitting for the orthosis 100 while simultaneously adding to the ease of donning and doffing (e.g., enabling single handed donning and doffing).

While the support strap 106 is illustrated as coming up off the medial side of the foot (e.g., in FIGS. 1A and 1B) and spiraling around the tibia to the lateral side of the leg and around the back of leg, it would be readily appreciated by one of ordinary skill given the contents of the present disclosure that the support strap 106 may be positioned in other configurations. For example, the user may position the support strap 106 above the foot plate 114 in a medial configuration, above the foot plate 114 in a lateral configuration, below the foot plate 114 in a medial configuration, or below the foot plate 114 in a lateral configuration depending on the patient's specific needs. The support strap 106 may also attach to the foot plate slots 118 from underneath the foot plate 114 and spiral up on either the medial or lateral side of the foot. For example, support strap 106 may attach to the foot plate slots 118 underneath the foot plate 114 on the lateral side of the foot, cross under the foot plate 114 and then cross up over the foot medially. Such a configuration may pick up the medial arch by spiraling across the lower leg and being anchored to the adjustable calf strap 102 on the lateral side of the calf.

The strapping system for the AFO 100 may be modified to control dorsiflexion, plantar flexion, as well as various varus and valgus deformities and/or other instabilities. Conceptually, the distances between different portions of the human anatomy change during the gait cycle. For example, the toe and heel may extend and contract relative to the shin during a step. These differences in length can be used with strap adjustments to provide tension and/or support for different foot movements (as opposed to a rigid support). For example, if the support strap 106 fully spirals around the leg of the wearer (see e.g., FIG. 1A), the support strap 106 will tighten in both plantar flexion as well as dorsiflexion, with a minimal amount of tension when the foot is in a relaxed position. However, if the support strap 106 does not fully spiral around the leg of the wearer, the support strap 106 will tighten during plantar flexion and will loosen during dorsiflexion. These and other configurations can be readily modified dependent upon the needs of the wearer. For example, if the support strap 106 comes up medially on the foot and only stays in front of the leg, the support strap 106 is controlling a valgus condition and supporting plantar flexion. If the support strap 106 comes up laterally on the foot and only stays in front of the leg, the support strap 106 is controlling a varus condition and supporting plantar flexion. If the support strap 106 comes up medially on the foot and spirals around the leg so that it covers both the front and back of the leg, the support strap 106 is controlling a valgus condition as well as supporting both plantar flexion and dorsiflexion. If the support strap 106 comes up laterally on the foot and spirals around the leg so that it covers both the front and back of the leg, the support strap 106 is controlling a varus condition as well as supporting both plantar flexion and dorsiflexion.

While specific AFO variations have been contemplated, it would be readily appreciated by those in the field that the novel elements of foot plate slots, a bottom strut dual arm, and a top strut arm, individually, or in combination, may be readily adapted for use in other AFO variations.

Referring now to FIGS. 1D and 1E, one exemplary ankle-foot orthosis (AFO) 100 is shown. This AFO 100 is similar to the aforementioned AFO 100 shown in FIGS. 1A-1C, but further comprises a D-ring 120 as the support strap 106 anchor point and may or may not have foot plate slots 118. The D-ring 120 may be positioned on the lateral or medial side of the foot plate 114. Furthermore, the D-ring 120 may be integrated into the foot plate 114 as part of one solid piece, or the D-ring 120 may be attached to the foot plate through screws, adhesives, or other suitable means. Alternatively, the D-ring 120 may be attached to the foot plate 114 with a hinge mechanism that allows the configuration of the D-ring to be adjusted between an above-the-foot plate configuration or a below-the-foot plate configuration. In some variations, the D-Ring 120 may be manufactured from a heat-moldable plastic, metal, leather, a carbon composite material, or various combinations of the foregoing.

The support strap 116 may be anchored to the D-Ring 120 by having an end section of the support strap 116 loop through the D-ring 120 and reattach to itself. The end section of the support strap 106 may be a permanent feature that is not removable from the D-Ring 120 or the end section of the support strap 106 may be reconfigurable and removable from the D-Ring 120 by using, for example, a hook and loop fastener (e.g., Velcro®), a clasp, a button, and/or any other suitable type of fastening mechanisms. This anchoring arrangement may allow multiple unique support strap configurations. For example, the user may position the support strap 106 above the foot plate 114 in a medial configuration, above the foot plate 114 in a lateral configuration, below the foot plate 114 in a medial configuration, or below the foot plate 114 in a lateral configuration depending on the patient's specific needs. In another variation, the user may position the support strap 106 in between the foot plate 114 and the insole 122.

FIG. 1E shows an AFO variant which includes the D-ring 120 and an insole 122 in combination with the foot plate 114 and the support strap 106. The insole 122 may include slots on its underside that are sized to accommodate the width of the support strap 106. The insole 122 may provide benefits by securing the position of the support strap 106 when, for example, the patient inserts his foot into a shoe while wearing the AFO. The insole 122 may also provide a flat surface for the wearer of the AFO 100, thereby minimizing discomfort associated with the support strap 106 being placed between the foot of the patient and the foot plate 114 of the AFO 100. In the variant depicted in FIG. 1E, the insole 122 may accommodate both varus and/or valgus deformity variants of the support strap 106. In some variations, the insole 122 may be manufactured from a heat-moldable plastic, metal, leather, a carbon composite material, or various combinations of the foregoing. In other variations, the support strap 106 can be positioned below the foot plate entirely.

While specific AFO variations have been contemplated, it would be readily appreciated by those in the field that the novel elements of a D-ring, an insole, a bottom strut dual arm, and a top strut arm, individually, or in combination, may be readily adapted for use in other AFO variations.

Ankle-Foot Orthoses with Angular Adjustments

As a brief aside, key outcomes of sequential orthotic treatment for pediatrics include a less restrictive orthosis like a foot orthosis (FO); if this is unsuccessful within a set timeframe, then the patient may require a more restrictive form of treatment such as an ankle-foot orthosis (AFO). Current research suggests that failure to tune ankle-foot orthosis and footwear combinations can lead to immediate detrimental effect on function, and, may actually contribute to long term deterioration. Recent studies have shown that only −50% of participants use ankle-foot orthosis/footwear combination tuning as standard clinical practice. The inclusion of an angular adjustment mechanism that is easy to adjust on the spot may be more convenient for the clinician to use, which may allow clinicians to provide better patient experiences and results.

Various aspects of the present disclosure are directed to ankle-foot orthoses that offer a plurality of different angular orientations of the foot plate relative to a patient's neutral stance. In one exemplary embodiment, a first strut arm is anchored to the patient's knee and mates to a second strut arm that is anchored to the foot plate.

In one exemplary embodiment, the mating allows the user to select an angular offset from among multiple possible angular positions. Functionally, the angular positions provide an angular offset from a neutral position (i.e., where the coronal, sagittal, and axial planes of the knee and the foot plate are parallel). In one specific embodiment, the angular offset may be relative to the axial plane; in other words, the toe of the foot plate may be angled up (dorsiflexion) or angled down (plantar flexion). More generally however, the angular offset may be with reference to any of the other planes e.g., the foot plate may be angled such that the medial edge of the foot is most distal (inversion), or such that the lateral edge is most distal (eversion).

While “stiffness” typically refers to the extent to which an object resists deformation in response to an applied force, orthotic fitting often must consider the perceived stiffness for the patient. Notably, the patient's central nervous system controls the musculoskeletal system during locomotion and the senses (visual, vestibular, proprioceptive, and tactile) provide important feedback related to gait. Impairment to any of these systems may affect the patient's gait, comfort, and/or orthotic usage. Since each patient may have different types of instabilities, the described straps and structures can modify the AFO for the patient's specific instabilities.

As used herein, “gait” refers to the pattern of limb movements made during locomotion. For bipedal human motion, a step is half of the gait cycle-a stride is a complete gait cycle (e.g., where the feet regain their initial relative position). Each step may be further divided into a “stance phase” (which may include initial contact, loading response, mid-stance, terminal stance), and a “swing phase” (which may include pre-swing, initial swing, mid-swing, and terminal swing). Normal gait should provide efficient locomotion and balance; abnormal gait may result in imbalance and/or inefficient locomotion. Generally, energy efficiency is measured based on the amount of vertical center of mass (COM) excursion during the gait cycle. Physiologically, gait may be affected by e.g., pelvic rotation, pelvic tilt, knee flexion during the stance phase, foot and ankle motion, knee motion, and lateral pelvic displacement.

Notably, adjusting the angular offset of the foot plate relative to the patient's knee affects the point at which the foot plate strikes the ground during the patient's step, as well as the stance and swing for the entire gait cycle. For example, a foot plate that is angled such that the toe strikes first, may allow the foot plate to flex slightly before the strut arms are loaded. This may result in less perceived stiffness and more spring. In contrast, a foot plate that is angled such that the heel strikes first may immediately transmit the foot impact with less/no flex. AFOs that enable convenient adjustment of the angular offset can be used by clinicians and patients to tailor the AFO to the patient's preferences and pathologies; e.g., a patient may need to feel the foot strike to a greater or lesser degree for comfort and/or gait stability.

Referring now to FIG. 1F, one exemplary ankle-foot orthosis (AFO) 100 is shown. This AFO 100 is similar to the aforementioned AFO 100 shown in FIGS. 1A-1E, but further comprises an angular adjustment gear 124. The angular adjustment gear 124 may comprise two separate gear pieces, wherein one gear piece may be integrated into the top section of the bottom strut dual arm 109 and another gear piece may be integrated into the bottom section of the top strut arm 10. The integration of the angular adjustment gear 124 may require the top section of the bottom strut dual arm 109 to have a circular opening at the center of the gear 124, wherein the radius of this opening is equal to or smaller than the inner radius of the gear. The integration of the angular adjustment gear 124 may require the bottom section of the top strut arm 110 to have a circular opening at the center of the gear 124, wherein the radius of this opening is equal to or smaller than the inner radius of the gear.

Each gear piece may comprise a number of gear teeth protruding from a strut arm, wherein each gear tooth may have an inner edge that extends to an inner circle, an outer edge that extends to an outer circle, a left edge that extends to one line of an equiangular set of lines extending radially from the center of the gear piece to the outer circle, and a right edge that extends to an immediately adjacent line of an equiangular set of lines extending radially from the center of the gear piece to the outer circle. These gear teeth edges may be perpendicular to the plane of the strut arm they protrude from, and the gear teeth may have a surface that is parallel to the to the plane of the strut arm they protrude from. The gear teeth are separated by an empty space that occupies an area between two lines of the equiangular set of lines extending radially from the center of the gear piece to the outer circle. Each gear piece may have between 2-100 gear teeth, preferably between 10-70 gear teeth, and more preferably between 30-50 gear teeth. Both gear pieces may have identical gear tooth dimensions. The angular adjustment gear 124 may provide a 360-degree range of the top strut arm 110 relative to the bottom dual strut arms 109 in increments equal to the circular pitch of the gear pieces. This gear tooth arrangement may permit the gear pieces to lock into each other in a mated configuration wherein the gear teeth of one gear piece fit into the empty spaces of the other gear piece, and vice versa. The integration of an angular adjustment gear into the strut arms has the added benefit of reducing the number of parts of the AFO 100.

The gear pieces may be held together in a mated configuration through the frictional forces between the gear teeth of the gear pieces. These frictional forces may be sufficiently strong to hold the gear pieces firmly together during normal wear of the AFO 100 and may be sufficiently weak to permit the wearer of the AFO 100 to separate the gear pieces into a non-mated configuration (e.g., to adjust the stiffness of the AFO 100 by changing the angle in the axial plane.)

The gear pieces may alternatively be held together through fastening mechanisms, including, but not limited to, a Chicago screw 130 or conventional nuts and bolts. A female portion of the Chicago screw 130 may be inserted through the opening at the center of the gear piece at the top section of the bottom strut dual arm 109 and further through the opening at the center of the gear piece at the bottom section of the top strut arm 110 until the screw's flange is flush with the outer side of the bottom strut dual arm 109. The gear pieces may then be mated in any configuration, securing the position of the top strut arm 110 relative to the bottom strut dual arm 109. A male portion of the Chicago screw 130 may be screwed into the female portion of the Chicago screw 130 that is inserted through both strut arms and tightened until the screw's flange is flush with the inner side of the top strut arm 110, locking the strut arms in place.

While a specific AFO variation has been contemplated, it would be readily appreciated by those in the field that the novel element of an angular adjustment gear may be readily adapted for use in other AFO variations.

Referring now to FIG. 1G, one exemplary ankle-foot orthosis (AFO) 100 is shown. This AFO 100 is similar to the aforementioned AFO 100 shown in FIG. 1F but further comprises a visual indicator 132. The visual indicator 132 may allow the patient or clinician to select a desired angle between the struts. The indicator may list the angles around the angular adjustment gear that define the angular adjustment. The visual indicator 132 may be lettering, notches, or symbols etched into the surface of the gear pieces or the struts that the gear pieces are incorporated into. The visual indictor may alternatively be printed, painted, molded, or otherwise written onto the surface of the gear pieces or the struts that the gear pieces are incorporated into.

While a specific AFO variation has been contemplated, it would be readily appreciated by those in the field that the novel element of a visual indicator 132 may be readily adapted for use in other AFO variations.

Referring now to FIG. 1H, one exemplary ankle-foot orthosis (AFO) 100 is shown. This AFO 100 is similar to the aforementioned AFO 100 shown in FIG. 1F or FIG. 1G, but the top strut arm 110 may have at least one indentation 134 that permits the attachment of at least one spring element 136. A first indentation may start 3 inches from the bottom of the top strut arm 110, may end 6 inches from the bottom of the strut top arm 110, and may be half an inch deep. A second indentation may mirror the first indentation and be positioned across from the first indentation. The indentation or indentations 134 may alternatively be positioned at any point along the top strut arm 110 that does not inhibit its functionality and the indentation or indentations 134 may be deep enough to enable the placement of at least one spring element 136. The indentation or indentations 134 may have attachment points that enable the attachment of at least one spring element 136. These attachment points may comprise a hook and loop fastener element (e.g., Velcro®), a clasp, a button, screws, and/or any other suitable type of fastening mechanisms. In another variation, these attachment points may be used to attach an overmold covering or a plastic casing or tubing, wherein the overmold covering or plastic casing or tubing snaps around or encloses the strut. This overmold covering or plastic casing or tubing may house at least one spring element 136.

The spring element 136 may comprise a spring or a part with spring functionality that applies an appropriate counter force when under either tension or compression. Alternatively, the spring element 136 may only apply an appropriate counter force when under only tension or only compression. While the illustrated spring element 136 is shown as a material spring (e.g., carbon fiber), any spring-like element (e.g., a coil spring, compression spring, tension spring, etc.) could be substituted with equal success. The spring element 136 may have at least one attachment point that enables the spring element 136 to attach to the top strut arm 110. The attachment points of the spring element 136 and the top strut arm no may be positioned in such a way as to have the spring element 136 sit within at least one indentation 134. The attachment point or points of the spring element 136 may comprise a hook and loop fastener element (e.g., Velcro®), a clasp, a button, screws, and/or any other suitable type of fastening mechanisms. In another variation, the spring element 136 may be attached to an overmold covering or a plastic casing or tubing.

The spring element 136 may be a permanent feature of the top strut arm 110 or the spring element 136 may be removable. The spring element 136 may be adjustable such that its response to tension, compression, or other forces may be changed. This adjustment may be performed while the spring element 136 is attached to the top strut arm no or if the spring element 136 has been removed.

Referring now to the leftmost variation in FIG. 1N, the AFO 100 may further comprise an adjustment mechanism 135. This adjustment mechanism 135 may be a clamp that has two openings located on either side of the clamp. Each opening may hold a spring element 136. The adjustment mechanism 135 may be positioned within the indentations 134 of the top strut arm 110. The openings may be positioned apart from each other at a distance that increases the tension of the spring elements 136. In another variation, the openings may be positioned apart from each other at a distance that decreases the tension of the spring elements 136. In yet another variation, the openings may be positioned apart from each other that manipulates the tension of the spring elements 136 in any desired way. In other variations, the adjustment mechanism 135 and any openings of the adjustment mechanism 135 may be shaped or positioned in any way to manipulate the spring elements 136 in any desired way. In other variations, the adjustment mechanism may be a band. In other variations, the adjustment mechanism 135 may be comprised of carbon composite disc springs. In yet other variations, the adjustment mechanism is any mechanism that may be positioned within the indentations 134 of the top strut arm 110 and that manipulated the tension or other properties of the spring elements 136.

The adjustment mechanism 135 may be removeable and interchangeable. Different adjustment mechanisms 135 with different stiffnesses may be applied to the AFO 100, providing variable stiffness in the strut arm no. The clinician or patient may attach different adjustment mechanisms 135 onto the AFO 100 to meet individual patient stiffness needs. In one specific implementation, the spring elements may be encased within an overmold so that they may be easily inserted, removed, and/or replaced without specialized tools.

Referring now to the central variation in FIG. 1N, the adjustment mechanism 135 may be positioned within the indentations 134 near the top of the indentations 134. This configuration may cause the spring elements 136 to change their tension or to take on different properties compared to if the adjustment mechanism 135 were positioned centrally within the indentations 134. The adjustment mechanism 135 may be used to manipulate the properties of the spring elements 136 by changing its position within the indentations 134.

Referring now to the rightmost variation in FIG. 1N, the adjustment mechanism 135 may have tightening and loosening functionality, wherein the width of the adjustment mechanism 135 may be increased or decreased. This increase or decrease in the width of the adjustment mechanism 135 may cause the spring elements 136 to change their tension or to take on different properties compared to if the adjustment mechanism 135 were in its neutral state. The adjustment mechanism 135 may be used to manipulate the properties of the spring elements 136 by adjusting the width of the adjustment mechanism 135.

While a specific AFO variation has been contemplated, it would be readily appreciated by those in the field that the novel elements of indentations 134, an adjustment mechanism 135, and spring elements 136 may, individually, or in any combination, be readily adapted for use in other AFO variations.

Referring now to FIG. 1I. one exemplary ankle-foot orthosis (AFO) 100 is shown. This AFO 100 is similar to the aforementioned AFO 100 shown in FIG. 1F, FIG. 1G, or FIG. 1H, but further comprises an angular adjustment socket mechanism 138 in place of an angular adjustment gear 124. The angular adjustment socket mechanism 138 may have a socket hub 140 positioned at the top of the bottom strut dual arm 109. The socket hub 140 may be integrated into the bottom strut dual arm 109 as part of a single piece or the socket hub 140 may be attached to the bottom strut dual arm with screws, adhesives, or other suitable means.

The socket hub 140 may comprise at least two sockets. The number of sockets in the socket hub 140 may be between 2-100, preferably between 2-30, more preferably between 2-15, and most preferably between 2-10. The socket openings may be circular, hexagonal, square, star-shaped, or any other common socket shape. The sockets may have inner threading to permit outside objects with screw threading to be screwed into the socket hub 140. The sockets may alternatively have an inner magnetic element to permit outside objects with a corresponding magnetic element to be held in place inside of socket hub 140 through magnetic forces. The sockets may alternatively have other common mechanisms to firmly hold an outside object in place, such that the outside object is removable.

The sockets on the socket hub 140 may be arranged in any manner that permits, using any of the sockets, the connection of a top strut arm 110 to the bottom strut dual arms 109. The socket openings may be arranged along a line wherein the openings are equidistant from each other, and the sockets are angled outwards equiangular from each other. The sockets on the socket hub 140 may have any depth, but preferably between 1-50 mm deep, more preferably between 1-25 mm deep, and most preferably between 10-25 mm deep. The sockets on the socket hub 140 may have any width, but preferably between 1-20 mm wide, more preferably between 1-10 mm wide, and most preferably between 3-10 mm wide.

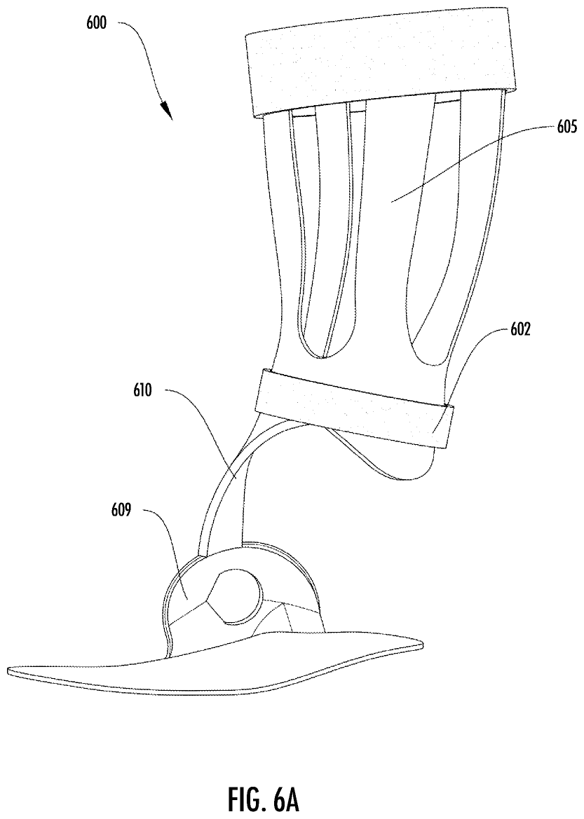

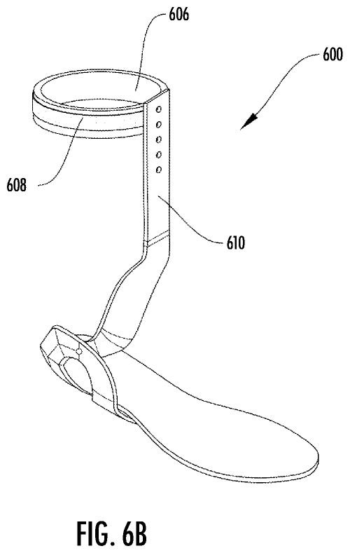

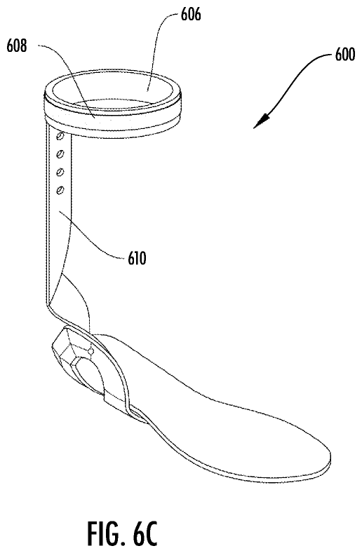

This arrangement of sockets may permit a wearer of the AFO 100 to change the angle in the axial plane by changing which socket the top strut arm no is connected into (e.g., so as to adjust the stiffness of the AFO.) In some variants, the angular adjustment socket mechanism may also provide the ability to fit both posterior and anterior AFOs (e.g., see below: Ankle-Foot Orthoses with Anterior/Posterior Support, and FIG. 6). In one such case, the foot plate 114 and the bottom strut dual arms 109 going around the ankle enable a user to change the top arm to either a posterior or anterior strut. Having an AFO that can support either anterior or posterior fittings may reduce inventory requirements for clinicians.

While specific AFO variations have been contemplated, it would be readily appreciated by those in the field that the novel element of an angular adjustment socket mechanism 138, may be readily adapted for use in other AFO variations.

Referring now to FIG. 1J, one exemplary ankle-foot orthosis (AFO) 100 is shown. This AFO 100 is similar to the aforementioned AFO 100 shown in FIG. 1F, FIG. 1G, FIG. 1H, or FIG. 1I but further comprises an angular adjustment bumper mechanism 142 in place of an angular adjustment gear 124. The angular adjustment bumper mechanism 142 may have a bumper hub 144 positioned at the top of the bottom strut dual arm 109. The bumper hub 144 may be integrated into the bottom strut dual arm 109 as part of a single piece or the bumper hub 144 may be attached to the bottom strut dual arm with screws, adhesives, or other suitable means.

The bumper hub 142 may comprise a circular strut portion 144 that encloses a cavity 148. The bumper hub 144 may be attached to the bottom strut dual arm 109 at the circumference of the circular strut portion 146. The bumper hub 144 may have an opening 150 at the top of the bumper hub 142, wherein the opening 150 permits the insertion of a top strut arm 110. A bumper cap 156 may be used to cover the bumper hub 142 and an enclosed bumper 156. The bumper hub 142, bumper 156, and bumper cap 152 may have an opening near the center of 23 each of the parts, wherein a bolt can be inserted through each of the parts, and wherein a nut can be tightened around the end of the bolt, locking the parts together.

The bumper hub 142 may have a bumper attachment point 154 located inside of the circular strut portion 144. A bumper 156 may be attached to the bumper attachment point 154, wherein the bumper 156 sits inside of the cavity 148 at the bumper attachment point 154. The bumper 156 may be any shape that holds the top strut arm at the desired angle. The shape of the bumper 156 limits the motion of the top strut arm 110, and, as a result, limits the rotation of the top strut arm 110 around the axial plane. The bumpers 156 may be removable and exchangeable for other bumpers 156. The bumpers 156 may have varying shapes that limit the rotation of the top strut arm 110 depending on the wearer's needs. The bumpers 156 may be color coded or stamped to indicate the amount of rotation that each bumper 156 permits. The bumpers 156 may be made from rubber, metal, or other common materials. The bumper 156 material may be chosen to provide a desired amount of stiffness for the top strut arm 110.

Referring now to FIG. 1L and FIG. 1M, one exemplary ankle-foot orthosis (AFO) 100 is shown. This AFO 100 is similar to the aforementioned AFO 100 shown in FIG. 1J but contains a different variation of the angular adjustment bumper mechanism. This angular adjustment bumper mechanism may require openings at the bottom of the top strut arm no and at the top of the bottom strut dual arm 109, wherein the openings allow the use of a Chicago screw 176 to hold the components of the angular adjustment bumper mechanism together, securing the top strut arm 110 relative to the bottom strut dual arm 109. This angular adjustment bumper mechanism may require further openings at the bottom of the top strut arm 110, wherein the openings allow screws 176 to inserted into the bumper hub 174.

This angular adjustment bumper mechanism may comprise a bumper hub 174 positioned at the outside of the bottom of the top strut arm no. The bumper hub 174 may be integrated into the top strut arm no as part of a single piece or the bumper hub 174 may be attached to the top strut arm no with screws, adhesives, or other suitable means. The bumper hub 174 may enclose the stationary bumpers 170 and the removable bumpers 172.

The angular adjustment bumper mechanism may further comprise a set of stationary bumpers 170 positioned on the inside of the top of the bottom strut dual arm 109. The stationary bumpers 170 may protrude outwards perpendicular to the inner surface of the top of the bottom strut dual arm 109 and be there may be 3 bumpers as part of stationary bumpers 170. In other variations, there may be between 2-10 bumpers as part of the stationary bumpers. Each bumper in the set of stationary bumpers 170 may take up a radial angle of 60 degrees. In other variations, each bumper in the set of stationary bumpers 170 may take up a radial angle of between 18-90 degrees. There may be gaps between the stationary bumpers 170 that allow the insertion of the removable bumpers 172 in between such gaps. These gaps may take up the same radial angle as the bumpers in the set of stationary bumpers 170.

The angular adjustment bumper mechanism may further comprise a set of removable bumpers 172. The removable bumpers 172 may be positioned around, and integrated into, a central hollow cylinder and there may be 3 bumpers as part of the removable bumpers 172. In other variations, there may be between 2-10 bumpers as part of the removable bumpers 172. Each bumper in the set of removable bumpers 172 may take up a radial angle of 60 degrees. In other variations, each bumper in the set of removable bumpers 172 may take up a radial angle of between 18-90 degrees. There may be gaps between the removable bumpers 172 that allow the removable bumpers 172 to be inserted into in between the stationary bumpers 170. These gaps may take up the same radial angle as the bumpers in the set of removable bumpers 170. In other variations, these gaps may take up a larger radial angle as the bumpers in the set of removable bumpers 170.

Each bumper in the removable bumpers 172 may have an opening, wherein this opening may have threading that allows screws 176 to be screwed in. Screws 176 may be inserted through the openings in the top strut arm no and screwed into each bumper in the removable bumpers 172, securing the removable bumpers 172 to the bumper hub 174.

An AFO 100 user may adjust the stiffness of the AFO 100 by replacing one set use removable bumpers 174 with another set of removable bumpers 174. For example, as illustrated in FIG. 1M, going from the middle to the right of the figure, the radial angle of each bumper in the second set of removable bumpers is 5 degrees lower than the radial angle of each bumper in the first set of removable bumpers.

Additionally, the different bumpers (and/or bumper hubs) may permit a user of the AFO 100 to change the angle in the axial plane by changing which set of removable bumpers 172 are used with the bumper hub 174 of the strut arm (e.g., to adjust the stiffness of the AFO.) In some variants, the removeable bumper/bumper hub mechanism may also provide the ability to fit both posterior and anterior AFOs (e.g., see below: Ankle-Foot Orthoses with Anterior/Posterior Support, and FIG. 6).

While a specific AFO variation has been contemplated, it would be readily appreciated by those in the field that the novel element of removeable bumpers/bumper hub mechanisms may be readily adapted for use in other AFO variations.

Referring now to FIG. 1K, one exemplary ankle-foot orthosis (AFO) 100 is shown. This AFO 100 is similar to the aforementioned AFO 100 shown in FIG. 1F, FIG. 1G, or FIG. 1H, but further comprises an angular adjustment tooth mechanism 160 in place of an angular adjustment gear 124. The angular adjustment tooth mechanism 160 may have a tooth hub 161 positioned at the top of the bottom strut dual arm 109. The tooth hub 161 may be integrated into the bottom strut dual arm 109 as part of a single piece or the tooth hub 161 may be attached to the bottom strut dual arm with screws, adhesives, or other suitable means.

The tooth hub 161 may comprise at least two tooth slot sets 162. Tooth slot sets 162 may start near the top of the tooth hub 161 and run from the circumference of the tooth hub 161 to near the bottom of the tooth hub 161. Each tooth slot set 162 may have between 1-10 tooth slots, preferably 1-6 tooth slots, and most preferably 4 tooth slots. Tooth slot sets 162 may run parallel to each other. The number of tooth slot sets 162 in the tooth hub 161 may be between 2-100, preferably between 2-30, more preferably between 2-15, and most preferably between 2-10. The tooth slot openings may be circular, hexagonal, square, star-shaped, or any other common slot shape. The tooth slots may have an inner magnetic element to permit outside objects with a corresponding magnetic element to be held in place inside of tooth hub 161 through magnetic forces. The tooth slots may alternatively have other common mechanisms to firmly hold an outside object in place, wherein the outside object is removable.

The tooth slot sets 162 on the tooth hub 161 may be arranged in any manner that permits, using any of the tooth slot sets 162, the connection of a top strut arm 110 to the bottom strut dual arms 109. The tooth slot openings may be arranged along a line wherein the openings are equidistant from each other and the tooth slots one tooth slot set are all parallel to each other. The tooth slots on the tooth hub 161 may have any depth, but preferably between 1-50 mm deep, more preferably between 1-25 mm deep, and most preferably between 10-25 mm deep. The tooth slots on the tooth hub 161 may have any width, but preferably between 1-20 mm wide, more preferably between 1-10 mm wide, and most preferably between 3-10 mm wide.

The top strut arm 110 may comprise a set of teeth 164 positioned at the bottom of the top strut arm 110. This set of teeth 164 may comprise between 1-10 teeth, preferably 1-6 teeth and most preferably 4 teeth. Each tooth may be covered with a thin rubber mold or bumper. The material for the tooth covering may be chosen to provide a desired amount of stiffness for the top strut arm 110.

The angular adjustment tooth mechanism 160 may include a slot covering 167 on the inside of the tooth slot sets 162, a tooth covering 165 on the outside of the teeth 164, or both. The slot covering 167 and the tooth covering 165 may be a layer of rubber. In other variations, the slot covering 167 and the tooth covering 165 may be a layer of other materials that have different stiffnesses. The inclusion of a slot covering, a tooth covering, or both may change the stiffness of the angular adjustment tooth mechanism 160, and, thus, the stiffness of the AFO.

This arrangement of teeth may permit a wearer of the AFO 100 to change the angle in the axial plane by changing which set of tooth slots 162 the set of teeth 164 of the top strut arm 110 is connected into (e.g., so as to adjust the stiffness of the AFO.) In some variants, the angular adjustment tooth mechanism may also provide the ability to fit both posterior and anterior AFOs (e.g., see below: Ankle-Foot Orthoses with Anterior/Posterior Support, and FIG. 6).

While a specific AFO variation has been contemplated, it would be readily appreciated by those in the field that the novel element of an angular adjustment tooth mechanism may be readily adapted for use in other AFO variations.

The foregoing discussion is presented in the context of a first strut arm and a second strut arm that mate according to various angular offsets, however artisans of ordinary skill in the related arts given the contents of the present disclosure will readily appreciate that other schemes for changing the angle of the AFO's foot plate strike may be used to address the patient's infirmities. For example, the footplate may include inserts in the toe or heel which result in a different foot plate strike; similarly, the foot plate may include both rigid, semi-rigid, and/or flexible materials to adjust the feel of the foot plate impact.

Additionally, while the foregoing discussion was presented in the context of the perceived stiffness as a function of the angular offset between strut arms, other mechanisms may be used to affect the energy return (“spring”), energy loss (“dampening”), and/or weight distribution of the AFO (“mass”) during each of the swing and stance phases. In one such example, the strut arms may be composed of different materials that impart different mechanical features (e.g., see below: Material Considerations for Ankle-Foot Orthoses Subcomponents). Other implementations may vary the length of the strut arms relative to one another to achieve different mechanical advantages. For instance, longer strut arms may provide more flex whereas shorter strut arms may transmit more torque (mutatis mutandis). Consequently, a proportionately longer strut attached to the foot plate may provide a more flex in the step, whereas a shorter strut attached to the knee cuff may allow for more energy transfer from the knee, or vice versa.

While the foregoing discussion has been presented with two strut arms, other implementations may use a greater or fewer number of strut arms. Additional strut arms may be used to add more degrees of freedom, improve support, or provide additional flexibility. For example, a two-strut assembly provides one degree of freedom; i.e., changing the foot plate angle also changes the distance between the tip of the foot plate and the knee cuff. A three-strut assembly may can be used to provide an additional degree of freedom over two strut arms; one degree of freedom may be used to create a desired angular offset and the second degree of freedom may be used to meet a specified overall length. In other words, a three-strut assembly may enable an AFO to support a large amount of foot plate angles and a large amount of leg dimensions. Similarly, a four-strut assembly may be used to support both sides of the foot rather than just the medial or lateral side. Even higher numbers of strut arms may be used to provide additional configurability; this may be particularly helpful in clinical settings where the brace may need to fit around/mount equipment (e.g., gait analysis labs may need e.g., camera mounts, accelerometers, gyroscopes and/or other sensors.)

Ankle-Foot Orthoses with “Hammock” Support

The foregoing discussion was presented in the context of straps that provide one-sided lateral or medial support during the gait cycle. However, some infirmities may require both lateral and medial support throughout the gait cycle; in these situations, an AFO may use a “hammock” or “hammock straps” on both medial and lateral sides of the foot plate. Functionally, the hammock straps may be adjusted to provide different amounts of tension (and support) for different foot movements on either/both sides of the foot throughout the gait cycle. As previously alluded to, adjusting strap locations can change the amount of strap material that is available to stretch/move. For example, strapping the medial side of the foot lower than the lateral side of the foot at rest may offer even support in the stance phase, but more medial support in the swing phase (i.e. the medial strap has less material and will tighten faster than the lateral side).

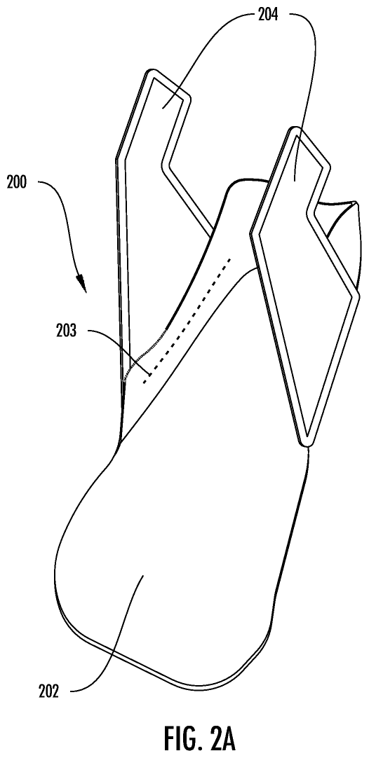

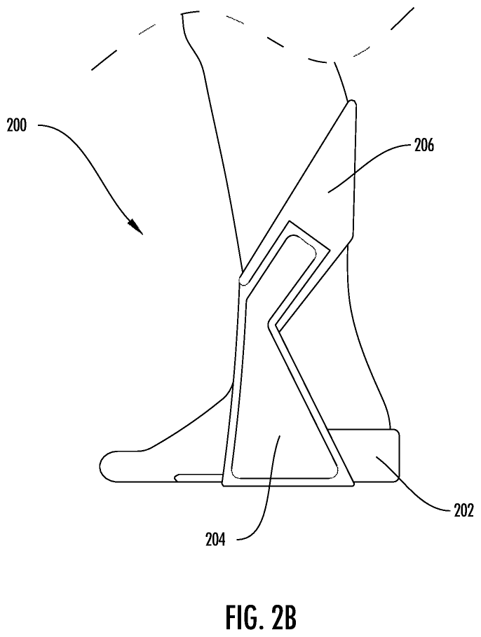

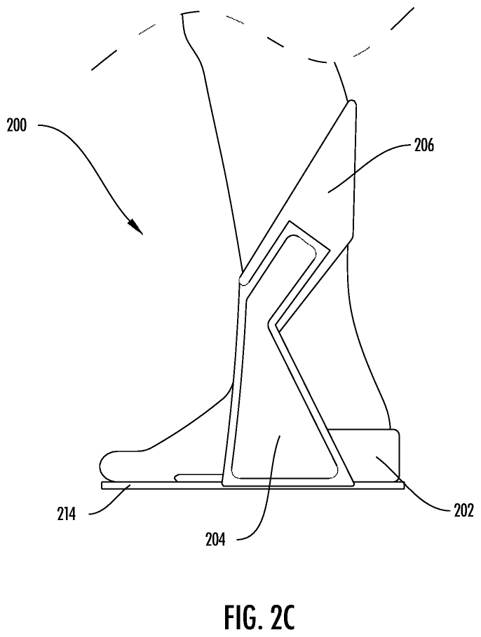

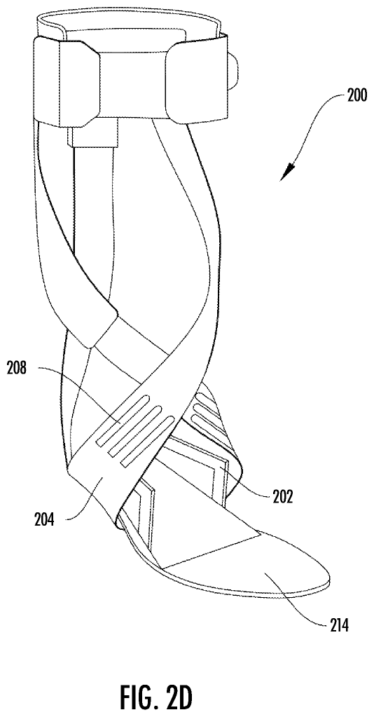

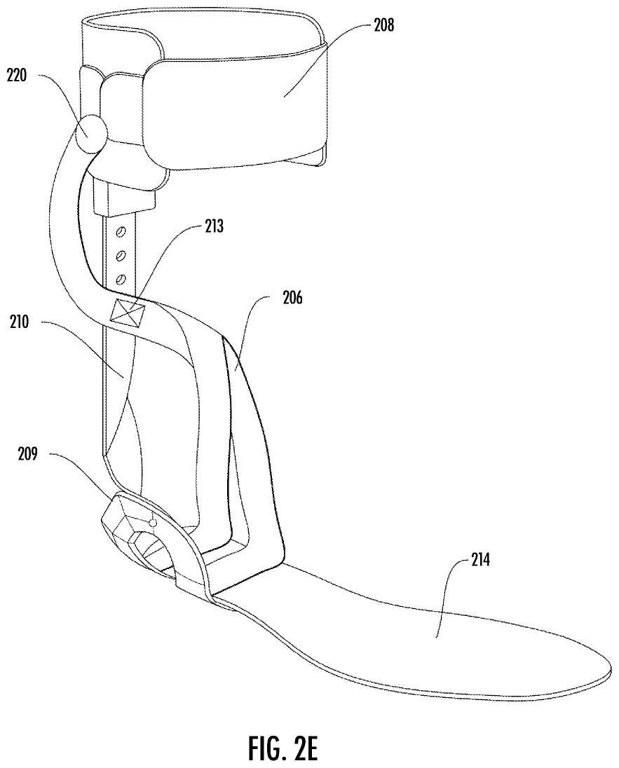

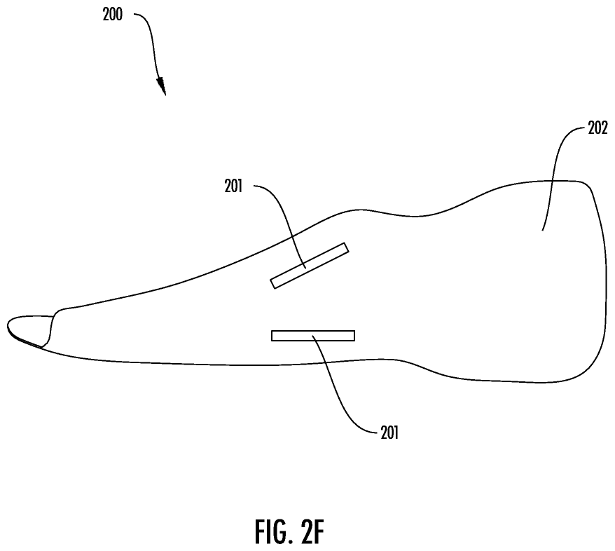

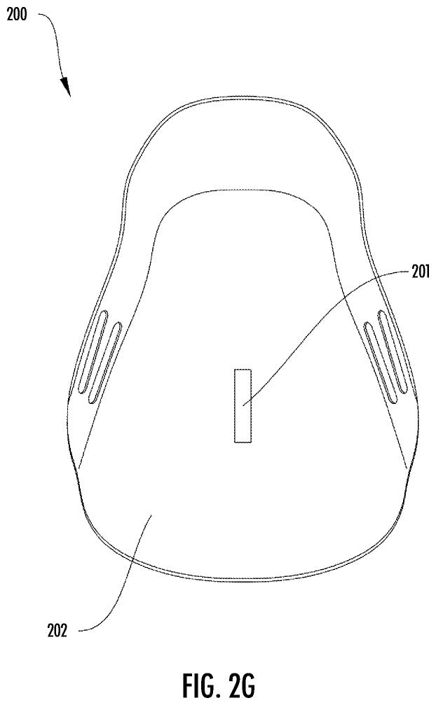

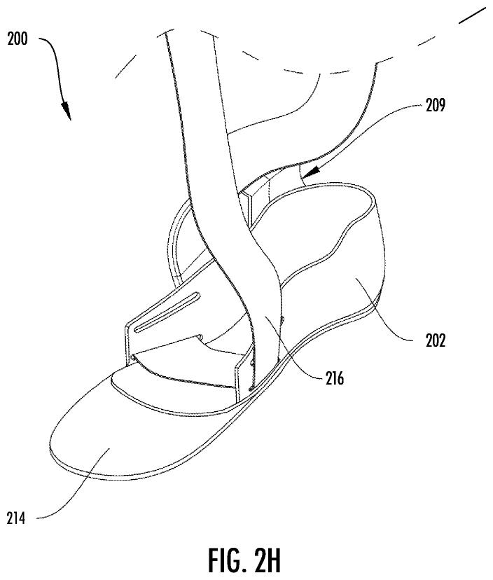

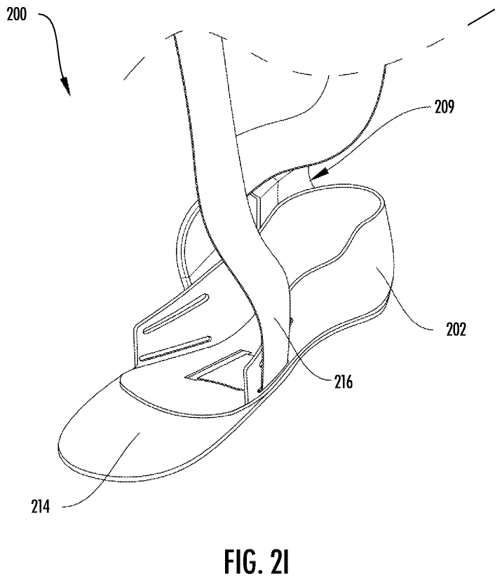

Referring now to FIG. 2A, one exemplary AFO 200 comprising an inner boot 202 and support straps 206, integrated together to form a “hammock” strapping system 204, is shown and described in detail. FIG. 2A illustrates an inner boot shell 202, its corresponding support straps 206, and an integration zone 203. The inner boot shell may be comprised of a rigid or semi-rigid material. This material may be a malleable thermoplastic that is molded into a boot shape during heat treatment or a very thin foam material for midfoot support and to help anchor the foot in the AFO 200. The material forming the inner boot shell 202 may have a thickness between 0.1-10 mm, preferably between 0.1-5 mm, and most preferably between 0.1-2 mm.

The inner boot shell 202 may first be formed over the mold of a patient's foot followed by the remainder of the AFO 200 structure, so the two pieces align correctly. In some cases, a foot that is unstable in the frontal plane may be braced with guiding structures around the foot. The guiding structures may offer a greater level of support around the foot plate. The thermoplastic molded inner boot that provides metatarsal support may be integrated or secured to a foot plate by permanently laminating the inner boot 202 into the carbon fiber, or securing it with sewing, stitching, hook and loop fasteners, snap buttons, or the like. In another variation, the inner boot 202 could be secured or attached to a foot plate through slots in the foot plate. In some variants, the inner boot 202 may have many readily available open slots to weave a strap into the boot. In another variation, the inner boot 202 may be attached to a foot plate through a D-ring. Still other implementations may laminate the plastic inner boot 202 into the carbon fiber; subsequent trimming may be used during fitting. The support straps 206 may be trimmed/cut-off if desired.

The inner boot shell 202 may have a bottom section that spans from the back of a wearer's heel towards the head of the wearer's metatarsal bones, providing metatarsal support. Additionally, the bottom section of the inner boot shell 202 may contain an arch that provides arch support. The inner boot shell 202 may have a back section and lateral and medial sections that extend upwards from the bottom section. The support straps 206 may be integrated with the inner boot shell 202 at the integration zone 203 to form a combined hammock strapping system 204. The inner boot shell 202 may have such integration zones 203 for the support straps 206 on the lateral and medial sections. In another variation, the inner boot shell may have an integration zone on the bottom section or on any other part of the inner boot shell 202. Such integration zones may comprise stitching, D-rings, foot plate slots, a clasp, a button, and/or any other suitable type of fastening mechanisms that corresponds to the attachment areas of the support straps 206. The inner boot shell 202 may be specifically manufactured to fit an individual wearer's foot or it may be manufactured in a set of standard sizes and designs.

The inner boot shell 202 may have a fabric material enclosing the heel area of the bottom section of the inner boot shell 202 as well as the lateral and medial sections of the inner boot shell 202. The fabric of the support straps 206 may further extend upwards beyond the inner boot shell, narrowing into two sections on either side of the wearer's foot. The hammock material may or may not have elastic properties.

Two support straps 206 may have attachment areas at the bottom section of the support straps 206. These attachment areas may comprise stitching, D-rings, foot plate slots, a clasp, a button, and/or any other suitable type of fastening mechanisms that corresponds to the attachment areas of the inner boot shell 202. The support straps 206 may be attached to the inner boot shell 202 at the attachment areas and the support straps 206 may be a removable or a permanent feature. The support straps 206 may be disposed around the wearer's leg and they may be positioned in a double strap configuration wherein the two support straps 206 cross over each other during their travel up the patient's leg. The top section of the support straps 206 may be anchored to a supporting structure such as the supporting structure 108 in FIGS. 1A and 1C.

While specific AFO variations have been contemplated, it would be readily appreciated by those in the field that the novel elements of an inner boot shell 202, a hammock strapping system 204, and support straps 206, individually, or in combination, may be readily adapted for use in other AFO variations.

Referring now to FIG. 2C, one exemplary AFO 200 is shown. This AFO 200 is similar to the aforementioned AFO 200 shown in FIG. 2A, but the support straps 206 sit below the boot 202 and above a foot plate, creating compression and lift.

While specific AFO variations have been contemplated, it would be readily appreciated by those in the field that the novel element of having the support straps 206 sit below the boot 202 and above a foot plate may be readily adapted for use in other AFO variations.

Referring now to FIG. 2D, one exemplary AFO 200 is shown. This AFO is similar to the aforementioned AFO 200 shown in FIG. 2A, FIG. 2B, or FIG. 2C, but further comprises elastic tension strings 208. The elastic tension strings 208 may be embedded inside of the hammock strapping system 204 or may sit on the outside of the hammock 204. The elastic tension strings 208 may be anchored at one end, at both ends, or at multiple points along the strings 208, wherein the anchor points may be anywhere on the inner boot shell 202, straps 206, foot plate 214, or any other area on the AFO 200. The AFO 200 may comprise between 2-1000 elastic tension strings 208, preferably 2-100 elastic tension strings 208, more preferably 2-10 elastic tension strings 208, and most preferably 6 elastic tension strings 208. The elastic tension strings 208 may comprise a single elastic material, a woven or non-woven combination of elastic materials, or a woven or non-woven combination of elastic and non-elastic materials.

The hammock strapping system 204 may have a fabric material enclosing the heel area of the bottom section of the inner boot shell 202 as well as the lateral and medial sections of the inner boot shell 202. The fabric of the hammock strapping system 204 may further extend upwards beyond the inner boot shell, narrowing into two strap-like pieces. These strap-like pieces may be disposed around the wearer's calf in a double-helix formation wherein the strap-like piece on medial side of the AFO 200 is disposed up and across the front of a patient's leg towards the lateral side of the AFO and the strap-like piece on medial side of the AFO 200 is disposed up and across the front of a patient's leg towards the medial side of the AFO. The top of these strap-like pieces may have attachment areas. The top end of the strap-like pieces may comprise a hook and loop fastener (e.g., Velcro®), a clasp, a button, and/or any other suitable type of fastening mechanisms. The top end of the strap-like pieces may be attached to each other, a supporting structure, an anchoring fabric, support straps, or any other attachment point.

While specific AFO variations have been contemplated, it would be readily appreciated by those in the field that the novel element of elastic tension strings 208 may be readily adapted for use in other AFO variations.

Referring now to FIG. 2E, one exemplary AFO 200 is shown. This AFO 200 may comprise a support strap 206, a supporting structure 208, a strut arm 211, an accessory attachment point 213, a foot plate 214, and a dial tensioning system 220. The support strap 206 may have a double strap formation, wherein a second strap is attached at its lower end near the bottom of the AFO 200 and attached at its upper end to the first support strap 206.

Solid AFOs often not only immobilize the ankle, which limits the ankle from moving into normal range of motion, but the rigid structure may also promote atrophy of the plantar flexors. There is a growing amount of evidence that the plantar flexors can create problems after a patient's stroke. For example, knee hyperextension (a common problem after stroke) was historically believed to be caused by spasticity in the plantar flexors or weakness in the quadriceps. More recently however, research has shown that knee hypertension is primarily the result of weakness in the plantar flexors and/or calf. Unlike existing solutions that focus on dorsiflexion (lifting the toes up), the described support strap 206 below allows plantar flexion (pointing the toes downward).

The support strap 206 may be attached at its lower end to the AFO 200 using corresponding hook and loop fastener pieces on the support strap 206 and AFO 200. In other variations, the support strap 206 may be attached at its lower end to the AFO 200 by having the lower end of the support strap 206 wrapped around a D-ring positioned along the AFO 200 such as in FIG. 1D or by having the lower end of the support strap 206 wrapped around foot plate slots on the foot plate 214 such as in FIG. 1A. In other variations, the support strap 206 is attached to points along a bottom strut arm.

The support strap 206 may wrap over the top of a patient's foot and then behind his or her calf but in front of the carbon fiber strut. The support strap 206 may then be attached at its upper end to a dial tensioning mechanism 220 positioned on the supporting structure 208. In another variation, a hook and loop or a buttonhole tightening system may be used to attach the upper end of the support strap 206 to the supporting structure 208.

The support strap 206 may have an attachment point 213, wherein the attachment point 213 is positioned such that the support strap 206 contacts the back of a wearer's calf. In one variation, the attachment point 213 may hold a removable pad 215 that cups the back of the wearer's calf. In another variation, the attachment point 213 is a wider portion of the support strap 206 that aids in cupping the calf. In another variation, the support strap 206 may include a removable stretchable fabric that goes around the calf; the stretchable fabric may be tightened via the upper support structure to provide lift and compression. This stretchable fabric may be a cylindrical, helically wound braid, for example, the common biaxial braid. In another variation, the support strap 206 may be integrated with a boot shell, such as the inner boot shell 202 of FIG. 2A.

While specific AFO variations have been contemplated, it would be readily appreciated by those in the field that the novel arrangement of the support strap 206, supporting structure 208, strut arm 211, accessory attachment point 213, foot plate 214, and the dial tensioning system 220, individually, or in combination, may be readily adapted for use in other AFO variations.

Referring now to FIG. 2F and FIG. 2G, one exemplary inner boot is shown and described. This inner boot is similar to the inner boot in FIG. 2A but may further comprise slots 201 in the inner boot shell 202. Each inner boot shell 202 may have at least one slot 201 and the slots 201 may be positioned laterally or medially on the sides of inner boot shells 200 as illustrated in FIG. 2F and FIG. 2G or centrally on the bottom of inner boot shells 200 as illustrated in FIG. 2G.

Referring now to FIG. 2H, one exemplary AFO 200 is shown and described. This AFO 200 is similar to the AFO in FIG. 1A but may further comprise an inner boot positioned over the foot plate 214. The inner boot may be secured to the AFO 200 by threading the support strap 216 through the slots 201 of the inner boot, wherein the support strap 216 originates at the foot plate slots, goes through the slot 201 on the bottom of the inner boot, goes through the slot 201 on the medial side or lateral side of the inner boot, and continues upwards until the support strap 216 is anchored to a dial tensioning system, supporting structure, or the like.

Referring now to FIG. 2I, one exemplary AFO 200 is shown and described. This AFO 200 is similar to the AFO in FIG. 1D but may further comprise an inner boot positioned over the foot plate 214. The inner boot may be secured to the AFO 200 by threading the support strap 216 through the slots 201 of the inner boot, wherein the support strap 216 originates at the D-ring, goes through the slot 201 on the lateral side of the inner boot, goes through the slot 201 on the medial side of the inner boot, and continues upwards until the support strap 216 is anchored to a dial tensioning system, supporting structure, or the like.

Having the multiple open slots 201 on the inner boot allows for many different strapping configurations. The ability to weave, tighten, or affix an inner boot to different spots on an AFO, in combination with the open slots on the foot plate and/or D-ring, makes the AFO modular. This modular design may allow the clinician to accommodate a patient's changing needs throughout the course of his or her treatment and to provide better patient experiences and results.

While specific AFO variations have been contemplated, it would be readily appreciated by those in the field that the use of slots 201 in an inner boot to secure it to an AFO 200 may be readily adapted for use in other AFO variations.

Ankle-Foot Orthoses with Integrated Gait Analysis

Gait analysis has historically been performed under laboratory settings with direct clinical supervision. Most gait analysis laboratories use imaging technology to capture and analyze a three-dimensional (3D) video of a person's gait. Pressure sensing floors may also be used to measure the foot strike during the gait. Gait analysis procedures usually include physical examination, video analysis, 3-D kinematic measures of joint motion, kinetic measures of forces that act across the joints during locomotion, postural balance analysis, and in some cases electromyographic (EMG) measurement of muscle activity during movement and/or other metabolic measurements. Usually, gait analysis begins with a physical examination to determine the degree of strength, range of motion, tone, and selective motor control in the lower extremity. Then the patient is recorded (video) standing and under locomotion; video cameras record and measure joint motion at the trunk, pelvis, hip, knee and ankle. The video data is used to assess the forces that cross the joints and characterize movement. In some cases, the patient may be required to wear an oxygen and heart rate monitor to assess metabolic efficiency.

Unfortunately, existing techniques for performing gait analysis are not accessible for most patient needs. Most patients purchase AFOs as part of an insurance plan. Due to health insurance billing and coding practices, many patients can only get a few re-fittings. Since gait analysis relies on very specialized equipment, there are very few clinical motion analysis laboratories in the United States. Gait analysis is expensive to perform and prescribed much less frequently than needed. This is particularly problematic for young children that are still learning to walk; uncorrected gait afflictions will affect them for the rest of their lives. Additionally, existing gait analysis techniques only provide a partial picture of the patient's needs; the controlled laboratory setting does not reflect the variety of everyday locomotion (uneven terrain, obstacles, twists/turns, and stride sizes, etc.) Different patients have different lifestyles which are often not reflected in the standardized testing procedure.

Various aspects of the present disclosure enable less expensive, more holistic gait assessment alternatives. In one embodiment, ankle-foot orthoses (AFOs) incorporate sensors, processors, and memory to record relevant data throughout the AFO's daily usage. In one exemplary embodiment, the collected data may be processed by the AFO itself or provided to a user application for gait analysis. Relatively simple check-ups may be performed in-home by patient care givers to ensure that patients are receiving the care they need. More sophisticated assessments may be done by clinicians, in some cases, remotely. The amount and quality of data collected over daily usage can provide a much more accurate assessment of the patient's gait and change over time (which for children may be quite significant).

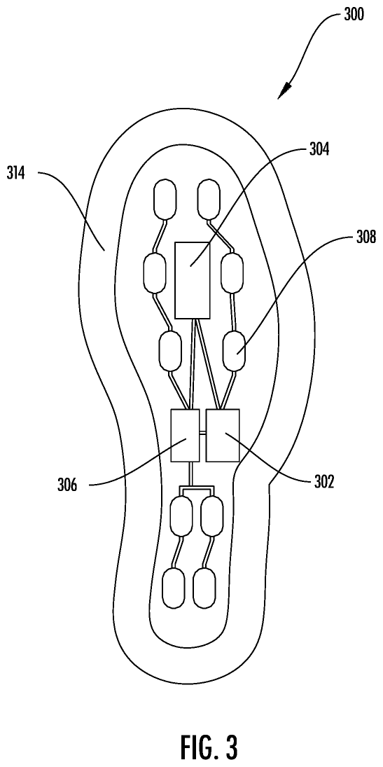

Referring now to FIG. 3A, one exemplary foot plate 300 is shown and described. The foot plate 300 may comprise an embedded programmable control module 302, battery pack 304, a communications module 306, and at least one pressure sensor 308.

In one exemplary embodiment, the AFO may include a time reference for time stamping data. Time stamped data may be used to time align measurements across different devices. For example, patients with infirmities in both legs can use both AFOs to capture their entire gait cycle. To ensure that both AFOs are time synchronized, the AFOs may periodically synchronize to each other, or a third independent time reference (e.g., a smart phone, smart watch, etc.). Additionally, while the present disclosure is presented in the context of the lower extremity, artisans of ordinary skill in the related arts will readily appreciate that the gait involves hips, torso, and arm movements as well. Relevant gait and/or metabolic data may also be collected on other devices (e.g., smart watches, brassieres, belts, etc.) for kinematic modeling and gait efficiency assessments. In one specific implementation, an AFO may be time synchronized to a smart phone or other video recording device; the patient can set the smart phone to record video of their foot motion while simultaneously capturing sensor readings from their AFO(s).

During operation, the pressure sensors measure the amount of pressure (force per unit area) at different locations for the sole of the foot. For instance, the pressure may be measured at the heel, ball, lateral edge and first “big” toe and fifth “pinky” toe. Notably, the rigid nature of the AFO and the patient's own control over their extremity may result in very different foot strike patterns compared to the sole strike patterns (e.g., a user that cannot control their forefoot may have an angular adjustment to their foot plate that exerts substantial pressure on the toe of the sole.) Consequently, some implementations may measure pressure for both the patient's foot and the sole of the foot plate. Pressure readings may be time stamped and stored “as-is”; in other implementations, pressure readings may be converted to a functional label (e.g., “pronated heel strike”, “supinated forefoot strike”, etc.)-functional labels may be suitable for reduced complexity processing/cursory analysis by the patient.

In one exemplary embodiment, the AFO may additionally include accelerometer(s) configured to measure changes in acceleration along one or more axis and/or gyroscope(s) configured to measure changes in rotation along one or more axis. The accelerometer(s) and/or gyroscope(s) may be distributed throughout the AFO to provide more granular assessments of motion at multiple joints. For example, an accelerometer and/or gyroscope may be located at each of the knee cuff, ankle, heel, medial toe edge and/or lateral toe edge.

In some variants, the AFO may be configured to collect data only for certain triggering conditions. Triggering conditions may be e.g., a time window, a duration, during activities of interest, for sensors of interest. As but one such example, a clinician/user may configure the type and amount of data collected over time by the AFO (e.g., a patient without knee issues may disable knee data collection, etc.) Reducing unnecessary data collection can reduce power consumption, increase measurement time, simplify processing complexity, and (for removeable sensors and batteries) reduce AFO weight.

The foot plate 300 may comprise an embedded programmable control module 302 that contains a processor that may be controlled by computer-executable instructions stored in memory to provide gait analysis functionality. In one exemplary embodiment, the programmable control module may be used to superimpose a force vector on a video sequence, negating the need to use an expensive gait laboratory. Alternatively, such gait analysis functionality may be provided in the form of an electrical circuit or be provided by a processor or processors controlled by computer-executable instructions stored in a memory coupled with one or more specially designed electrical circuits. Gait analysis may also be performed by an external device, wherein the foot plate 300 contains a communications module 306 with wired or wireless communication means that can transfer pressure sensor 306 data and other data to an external device. The external device may be 3D gait analysis equipment.

In one specific embodiment, a user application may be used by a clinician to record time synchronized video of a patient's gait. Pedometer, accelerometer, and/or gyroscope readings for the AFO may be used, along with the patient's limb dimensions, to infer the movement of the trunk, pelvis, hip, knee, and/or ankle. The motion data may be super-imposed over the video sequence to provide a visual representation of the patient kinematics; additionally, time synchronized pressure readings and patient weight distribution information can be used to infer forces that cross the joints and characterize movement. Notably, the patient limb dimensions, and weight distribution information may be performed as part of a physical examination. In other words, the exemplary AFO gait analysis can provide similar results using commonly available compute resources (e.g., a smart phone) to a gait analysis laboratory at substantially less cost.

In another specific embodiment, a user application may be used by the patient to record their day-to-day activities while wearing their AFO. Even though patients may be intimidated by large data sets, their clinician can pre-define functional labels to improve patient self-care. For example, a clinician may prescribe rehabilitation therapy to improve muscle strength; the AFO can be pre-configured with functional labels that identify conditions of interest. Every night, the patient can retrieve a daily log of their activity and peruse the functional labels to implement self-care. Large deviations in expected improvement may be a good reason to flag early re-assessment.

The foot plate 300 may comprise an embedded battery pack 304, wherein the battery pack 304 has the ability to be charged through inductive charging, enabling the foot plate to be entirely free of any openings for plugs or wire passages. Alternatively, the foot plate 300 may have a USB-C port that allows wired charging we well as wired communication between the communications module 306 and external devices.

The foot plate 300 may connect to an external device, peripheral, or accessory, e.g., a smartphone. This foot plate 300 may communicate with the external devices, peripherals, or accessories using standard wired or wireless communications connections. The foot plate 300 may contain an embedded pedometer that adds pedometer functionality to the foot plate.

In other variations, the electrical components and functionality may be integrated into an inner boot and other AFO components.

While specific AFO variations have been contemplated, it would be readily appreciated by those in the field that the novel foot plate elements of an embedded programmable control module 302, battery pack 304, a communications module 306, and at least one pressure sensor 308, individually, or in combination, may be readily adapted for use in other AFO variations.

Advantageously, embedded gait analysis capabilities may synergize particularly well with the universal strapping and adjustment capabilities described throughout. Notably, one-piece rigid AFOs cannot be adjusted-as a result, patients learn to walk with the AFO. This stilted gait may introduce new problems in locomotion over time. Unlike rigid AFOs, the exemplary strap-based and/or adjustable AFOs allow the user to modify their needed support. By monitoring their gait history, the user can make adjustments to improve muscle strength, muscle memory, and eventually mobility. As but one such example, a clinician may review pressure map data obtained from the AFO, and notify the patient to correctively adjust their angular offset. The patient may then adjust stiffness and/or strut arm angles based on gait history feedback, consistent with other portions of the present disclosure (see e.g., Ankle-Foot Orthoses with Angular Adjustments, above). In other examples, a patient may switch between lateral, medial, or hammock strapping and/or swap between anterior and posterior supporting structures according to their gait history feedback. Of particular note, the foregoing process may occur entirely remotely—the clinician may receive the pressure map data via email or similar file transfer. They may responsively notify the patient to make the adjustment, and verify the adjustment based on an updated pressure map data. The patient can make the adjustments at home, and does not need to set foot in the clinician's office.

As a practical matter, raw gait data may not be well understood by patients or even most therapists, thus certain embodiments may quantify gait according to one or two more easily understood metrics. For example, in one specific embodiment, the AFO may provide an average of the vertical center of mass (COM) excursion during the patient's gait cycle. This may be further simplified on a scale of e.g., 1-10, or similar indicia. The patient can adjust their fitting and/or gait to reduce vertical COM excursion (e.g., improving gait efficiency). Even if the patient cannot achieve a normal gait, minimizing vertical COM excursion reduces strain on the joints and can improve long term health outcomes.

Furthermore, the various techniques described herein may be broadly extended to other applications that utilize gait analysis. While most gait issues in adults are the result of long term/chronic health conditions (e.g., stroke, nerve trauma, and/or other nervous system impingement), children may have conditions that are operable. Where surgical intervention is possible, gait analysis may be prescribed for children a few months before surgery; in some cases, the surgeon may use gait information to guide the surgical intervention. Additionally, gait analysis may be prescribed post-operation, to assess the effectiveness of the surgical intervention.

While the foregoing discussion is presented in the context of a pressure sensors, accelerometers, and gyroscopes to capture “motion capture data”, artisans of ordinary skill in the related arts will readily appreciate that any suitable technique for measuring joint motion and forces during locomotion, balance, and/or muscle activity during movement, may be substituted with equal success. For example, the struts and/or support structure may incorporate pressure sensors and/or strain gauges to determine the amount of force exerted on the AFO. As another example, the AFO may incorporate microphones to measure sole strike frequency and/or intensity (certain foot strike patterns may be characterized by acoustic patterns).

In the aforementioned embodiments, motion capture data was time stamped according to a synchronized time reference. Alternative implementations may organize motion capture data based on gait-phase references; for example, pressure data from the user's foot can be used to demarcate stance phases for each step, e.g., the heel strike may correspond to initial contact, the ball and lateral edge may correspond to loading response, toe contact may correspond to terminal stance, etc. Similar approaches may use the accelerometer and gyroscope to demarcate swing phases for each step, e.g., heel lift through forefoot motion may be treated as pre-swing/initial swing, the foot's maximum are speed may indicate the mid-swing, and landing through heel strike may be the terminal swing, etc. Notably, the foregoing examples use different sensor modalities to discern gait phases, similar results could be performed using just pressure sensor data or accelerometer/gyroscope data. Reduced modality variants may e.g., simplify processing, reduce capture data sets, and/or minimize power consumption.

As previously alluded to, some gait analysis techniques attempt to measure energy efficiency or “metabolic data”. To these ends, some variants may incorporate sensors that e.g., measure heart rate, heart rate variability, blood oxygen, body temperature, perspiration, and/or electrical activity. In some such variants, the metabolic data may be time aligned to motion capture data.

Some patients may want to take a more hands-on approach to embedded gait analysis and monitoring. Thus, certain embodiments may additionally allow the patient to identify trigger conditions of interest to automate data collection. Examples may include data collection according to e.g., time of day (most energy, most fatigued), activity level, activity type, and/or other user identified criteria. Still other modes may allow for a “rolling capture” for unpredictable events, such as falls, trips, or other unplanned, but possibly important, events. During rolling capture, the AFO continuously captures motion data within a first-in-first-out (FIFO) short term memory buffer. The FIFO may only store a finite amount of data before it overwrites itself, however-in the event of an interesting event, the patient can trigger a memory dump to a separate long term memory. A user may then review only the accumulated long term events (e.g., only falls, trips, etc.)

Certain embodiments of the present disclosure may additionally enable external 3^{rd} parties to monitor the patient's usage and mobility data. Due to the sensitive nature of health monitoring data, the embedded programmable control module may include encryption, decryption, authentication and/or authorization algorithms. For example, a clinician, health insurer, or caregiver may be able to view motion capture data and/or metabolic capture data. In one specific implementation, the requesting 3^{rd} party may need to verify their access credentials and/or may only have limited access. This information may be used to verify that the patient is using the device correctly, adjust the device to fit lifestyle/gait and/or developmental changes, monitor changes to activity levels, and/or inform outcome-based bracing/treatment. In some cases, ongoing services may additionally incorporate subscription and/or per-use type monitoring. This may be used to meter access and/or ensure that billing and charging is consistent.

Ankle-Foot Orthoses with Universal Foot Plate

As a brief aside, the length and function of foot plates vary from patient to patient. While adults have established gait patterns, children are both physically growing and developing their gait. Unfortunately, orthotists often leave an extra ¼″ to ⅜″ of foot plate past the end of a child's toes to allow for growth. Leaving more than this would interfere with the child's gait development and may make it especially difficult to find shoes to wear over the braces. Thus, having a foot plate that can be continuously adjusted to the proper length may eliminate the need for the foot plate to be constantly replaced and decrease the likelihood that the child will develop an atypical walking pattern. More directly, adjustable foot plates may be crucial for child development because: (1) they affect how a child develops their adult walking pattern; (2) they affect how a child improves strength in feet and legs, (3) improper fit can adversely affect stability/balance while walking and standing, (4) proper fit can be used to correctly position the foot.

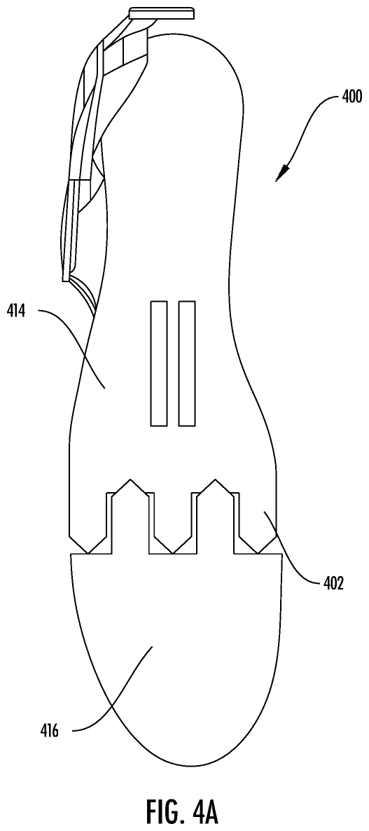

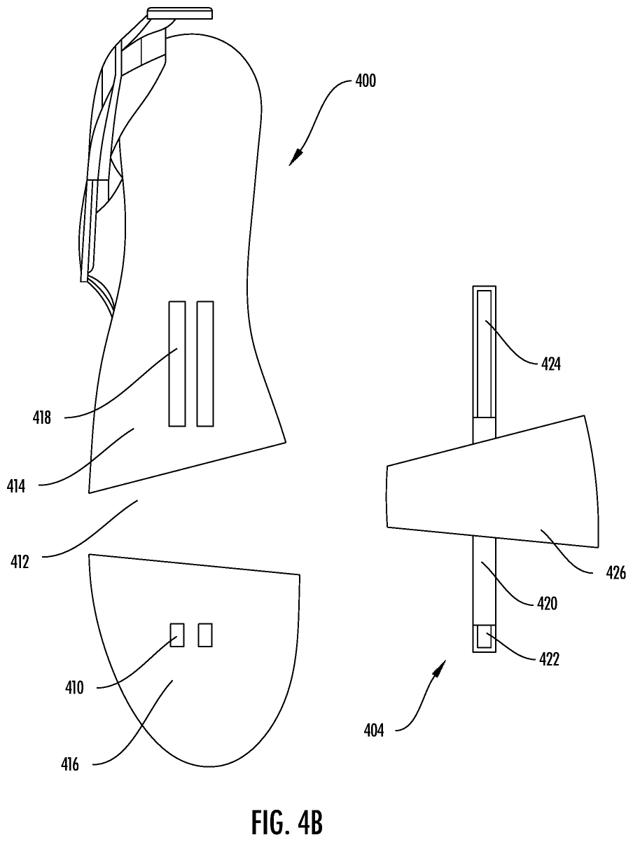

Referring now to FIG. 4A, one exemplary AFO 400 is shown and described. This AFO 400 comprises a length-adjustable foot plate that may be incorporated into any of the aforementioned AFOs. The length-adjustable foot plate may be comprised of an interlocking foot plate 414 and toeplate 416. The foot plate 414 may have at least one protrusion 402 that fits into at least one corresponding cavity in the toeplate 416. Likewise, the toeplate 416 may have at least one protrusion 402 that first into at least one corresponding cavity in the foot plate 414. The toeplate 416 may be joined tightly to the foot plate 414 by inserting the protrusions 402 of one foot plate piece into the cavities of the other foot plate piece. The protrusions 402 and corresponding cavities may be shaped to permit adjustment of the distance between the foot plate pieces along the heel-toe axis, allowing the wearer to increase or decrease the overall length of the combined foot plate. The interlocking foot plate 414 and toeplate 416 may have a locking mechanism, wherein the locking mechanism secures the foot plate 414 and toeplate 416 together into one combined foot plate. Gaps or empty spaces left in between the foot plate 414 and toeplate 416 may be filled in with inserts to prevent the formation of pressure points. The inserts may be made of rubber.

While specific AFO variations have been contemplated, it would be readily appreciated by those in the field that the novel elements of a foot plate 414, a toeplate 416, and protrusions 402, individually, or in combination, may be readily adapted for use in other AFO variations.

Referring now to FIG. 4B, another exemplary AFO 400 is shown and described. This AFO 400 comprises a length-adjustable foot plate that may be incorporated into any of the aforementioned AFOs. The length-adjustable foot plate may be comprised of a foot plate 414 with rear foot plate slots 418, a toeplate 416 with front foot plate slots 410, and a length-adjuster 404. The length-adjuster 404 may be comprised of a spine piece 420, a front protrusion 422, a rear protrusion 424, and an insert 426. The spine piece 420 may be generally flat and rectangular but may also take on any other shape that does not inhibit the functionality of the AFO 400. The foot plate 414 and the toeplate 416 may have slots or indentations on the bottom side that fit the entirety of the spine piece and help hold it in place. The length adjuster may be used as a slider to keep the length of the combined foot plate 414 and toeplate 416 in place.

The front protrusion 422 and the rear protrusion 424 may be integrated into the spine piece 420 and may be positioned on the bottom and top sections of the spine piece respectively. The front protrusion 422 may have a square or rectangular shape that corresponds to the square or rectangular shape of one of the front foot plate slots 410 and the front protrusion 422 may fit into one of the front foot plate slots 410, holding the spine piece 420 in place relative to the toeplate 416. The front protrusion 422 and front foot plate slots 410 may alternatively have any shape that permits the front protrusion 422 to fit tightly into the front foot plate slots 410 while not inhibiting the functionality of the AFO 400.

The rear protrusion 424 may have a square or rectangular shape that corresponds to the square or rectangular shape of one of the rear foot plate slots 418 and the rear protrusion 424 may fit into one of the rear foot plate slots 418, holding the spine piece 420 in place relative to the foot plate 414. If the spine piece 420 is connected to both the first and toeplates, the foot plate 414 is secured in place relative to the toeplate 416, creating a single rigid or semi-rigid foot plate structure.

The insert 426 may be generally flat and may be shaped to fill in any gap between the foot plate 414 and the toeplate 416. The shape of the insert 426 can be varied to ensure that the overall foot plate length is proper once the foot plate 414 and the toeplate 416 are combined with the length-adjuster 404. The insert 426 may be a permanent feature of the length-adjuster 404. In this case, the insert 426 may be an integrated part of the length-adjuster 404, forming a single piece, or the insert 426 may be permanently fixed onto the length-adjuster 404 using screws, adhesives, or other suitable means.

Alternatively, the insert 426 may be a removable feature of the length-adjuster 404. In this case, the insert 426 may be attached to the length-adjuster 404. When the foot plate length needs to be adjusted, the AFO 400 wearer may detached the old insert and attach a new insert to the length-adjuster 404, increasing or decreasing the length of the foot plate based on the size of the new insert.

While specific AFO variations have been contemplated, it would be readily appreciated by those in the field that the novel elements of a foot plate 414, a toeplate 416, and a length-adjuster 404, individually, or in combination, may be readily adapted for use in other AFO variations.

Ankle-Foot Orthoses with Anterior/Posterior Support

Referring now to FIG. 6A, one exemplary AFO 600 is shown and described. This AFO 600 comprises a bottom strut dual arm 609 and a semi-rigid calf cuff 602 that may be incorporated into many of the aforementioned AFOs. The upper strut formation 605 ensures that no pressure and/or heat builds up on the posterior or anterior sides of a wearer's foot and provides both anterior and posterior support. Furthermore, the incorporation of a semi-rigid calf cuff 602 will help the wearer maintain a functional position and improve his or her stability for successful standing and walking. In another variation, the upper strut formation 605 may be a clam shell with both sides fully encircling a patient's calf to create the same stability for the patient.

The semi-rigid calf cuff 602 may be generally hollow, short, flat, and cylindrical or elliptically cylindrical and may have thin walls. The type of material and the thickness of the material may be chosen to provide the semi-rigid calf cuff with semi-rigid properties. The semi-rigid calf cuff may be positioned along the top strut arm 610. The semi-rigid calf cuff 602 may be integrated into the top strut arm 610 or the semi-rigid calf cuff 602 may be attached to the top strut arm 610 through the use of screws, adhesives, or other common fasteners.

The semi-rigid calf cuff 602 may further comprise a fastening strap 604 wrapped around the outer circumference of the semi-rigid calf cuff 602. One end of the fastening strap 604 may be permanently attached to the semi-rigid calf cuff 602 at one point along the outer circumference of the semi-rigid calf cuff 602. The fastening strap 602 may be a hook and loop wrap wherein the loose end of the fastening strap 602 may be attached to the fastening strap 602 at one point along the outer circumference of the fastening strap 602. The fastening strap 602 may be adjusted by detaching the loose end of the fastening strap 602 from itself and reattaching the loose end of the fastening strap 602 onto a different point along the outer circumference of the fastening strap 602, wherein the circumference of the fastening strap 602 may be increased or decreased to adjust the semi-rigid calf cuff 602.

While specific AFO variations have been contemplated, it would be readily appreciated by those in the field that the novel elements of a bottom strut dual arm 609 and a semi-rigid calf cuff 602, individually, or in combination, may be readily adapted for use in other AFO variations.

FIGS. 6B and 6C depict an exemplary AFO 600 capable of anterior fitting (shown in FIG. 6B) and posterior fitting (shown in FIG. 6C). This AFO 600 is similar to the aforementioned AFO shown in FIG. 6A but substitutes a rigid calf cuff 606 in place of a semi-rigid calf cuff.

The rigid calf cuff 606 may be generally hollow, short, flat, and cylindrical or elliptically cylindrical and may have thin walls. The type of material and the thickness of the material may be chosen to provide the rigid calf cuff 606 with various material properties (stiffness, weight, comfort, hypoallergenic, etc.). The rigid calf cuff 606 may be positioned along the top strut arm 610. The rigid calf cuff 606 may be integrated into the top strut arm 610 or the rigid calf cuff 606 may be attached to the top strut arm 610 using screws, adhesives, or other common fasteners.

The rigid calf cuff 606 may be attached to an AFO that has an angular adjustment mechanism, wherein the rigid calf cuff 606 may be attached on the anterior (shown in FIG. 6B) or posterior (shown in FIG. 6C) of the top strut arm 610 based on how the user adjusts the angular adjustment mechanism. The added benefit of this arrangement is that a single brace may be used as either a posterior or an anterior brace; a common problem for small independent O&P clinics (orthopedic and prosthetic) is on-hand inventory. Stocking anterior braces and posterior braces in similar sizing takes significant amounts of space. A universal or modular brace that can provide both posterior and/or anterior support can reduce the clinic's on-hand inventory by half.

In one exemplary embodiment, angular adjustment mechanism between the bottom strut arm and the top strut arm may be any of e.g., gears, sockets, teeth, bumpers, and/or any similar mechanism (see above, Ankle-Foot Orthoses with Angular Adjustments and any of the corresponding FIGS. 1F-1M) For example, an angular adjustment gear may allow a wearer to quickly and easily transform the top strut arm 110 between an anterior and posterior position, as well as into many other possible positions. A supporting structure, such as the supporting structure 606, may be attached to the top strut arm 110 in an anterior position such as in FIG. 6B or in a posterior position such as in FIG. 6C. This versatile design of the angular adjustment gear 124 permits the AFO 100 to work as a universal AFO.

While specific AFO variations have been contemplated, it would be readily appreciated by those in the field that the novel element of the rigid calf-cuff 606 may be readily adapted for use in other AFO variations.

Ankle-Foot Orthoses with Multiple Adjustments

Rotary adjustment systems (such as a BOA® dial) are used in some AFOs to make gross fitting adjustments. Practically, this requires the AFO wearer to turn the BOA® dial with a ratchet over several turns-this may be difficult for patients that do not have dexterous hands (e.g., stroke survivors). Various aspects of the present disclosure are directed to a multiple adjustment scheme with gross adjustments at discrete fittings, and fine tune adjustments along a continuous range. In one exemplary embodiment, an AFO with button hooks and holes (or snaps) enables gross adjustments to the length of the support strap. The minor adjustments to the length of the support strap may be made with a rotary tensioning system (e.g., a BOA® dial tensioning system). In this case, both gross and fine adjustments can be made with only using one hand. The dual adjustments provides a technological solution that results in an AFO with a better fit, improved comfort, and easier adjustability.

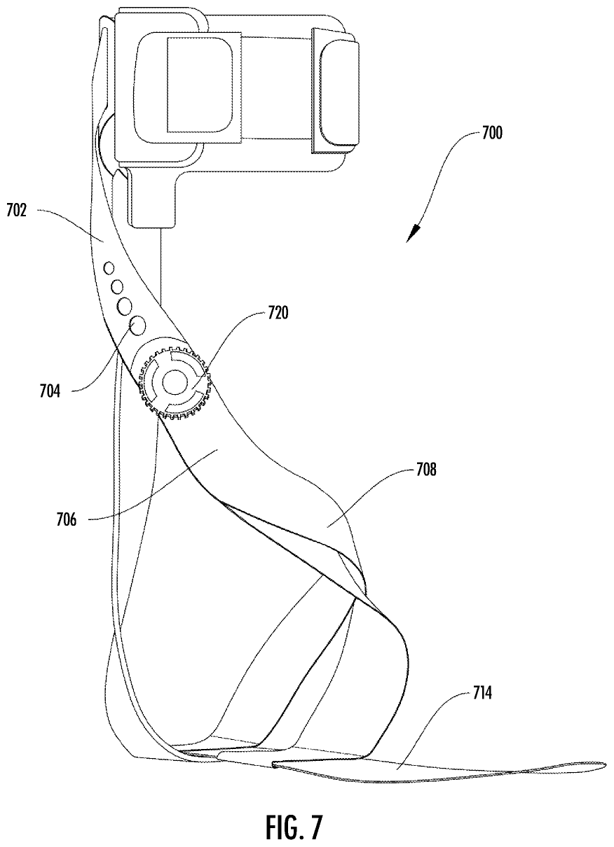

Referring now to FIG. 7, one exemplary AFO 700 is shown and described. This AFO 700 comprises a rotary tensioning system 720 (such as a BOA® dial), a support strap, button hook holes 704, and a button.

The support strap may comprise an upper portion 702, a center portion 706, and a lower portion 708. The lower end of the lower portion 708 of the support strap may be attached to a foot plate 714. The upper end of the lower portion 708 of the support strap may be attached to the rotary tensioning system 720. The lower end of the center portion 706 of the support strap may be attached to the rotary tensioning system 720. The length of the lower portion 708 and the center portion 706 of the support strap may be adjusted using the rotary tensioning system 720. The upper end of the center portion 706 of the support strap may have a button hook attached on the inside of the upper end of the center portion 706 of the support strap. The lower end of the upper portion 702 of the support strap may comprise button hook holes 704, wherein the button hook holes 704 are positioned at pre-determined spots along the length of the upper portion 702 of the support strap. The button hook on the inside of the upper end of the center portion 706 of the support strap may be inserted into the button hook holes 704, connecting the upper end of the center portion 706 of the support strap to the lower end of the upper portion 702 of the support strap.

In another variation of the AFO 700, there may be a button hook hole 704 on the upper end of the center portion 706 of the support strap and a series of button hooks along the upper portion 702 of the support strap.

In another variation of the AFO 700, the button hook holes 704 and the button hook may have corresponding magnetic elements that help guide the button hook into the button hook holes 704 and improve the strength of the connection. While specific AFO variations have been contemplated, it would be readily appreciated by those in the field that the novel use of a button hook and button hook holes 704 may be readily adapted for use in other AFO variations.

### “Sock-Like” Ankle-Foot Orthoses

One basic assumption that underpins existing AFO designs is that the ankle, foot, and knee should be stabilized according to a rigid configuration. As a result, existing AFOs use a rigid one-piece support that secures the ankle and foot in neutral alignment with the knee. Of particular note, the rigid support structures of the AFO must be designed to tolerate all types of mechanical stresses: e.g. compression, tension, shear, torsion, and bending. Additionally, the rigid support structures must be conservatively designed to handle even the most mundane day-to-day activities. For example, a rigid one-piece AFO must be designed to handle a substantial portion of the body's full weight during the gait cycle; this requires materials that have very high strength to weight ratios.

As used herein, the terms “rigid” and its derivatives, refer to a physical arrangement of components that does not deform during mechanical stresses for their intended operation. In other words, a rigid one-piece AFO transfers the rotations, translations, and/or other mechanical motion experienced by the foot plate to the knee cuff and vice versa. The terms “semi-rigid”, “flexible” and its derivatives, refer to a physical arrangement of components that reversibly deforms during mechanical stress. In other words, a semi-rigid foot plate may “flex” during usage, resulting in some lossy transfer of mechanical motion to the knee cuff and vice versa.

The strapping embodiments described in the present disclosure use tensioning techniques to enable meaningful support throughout a range of motion. Specifically, the strapping embodiments leverage the body mechanics during motion to create tensioned support that changes with different foot movements. Unlike rigid one-piece supports, tensioning cables only need to resist tension stresses; the straps do not need to prevent e.g., compression, shear, etc. This greatly reduces the material strength requirements of the straps. Furthermore, while rigid one-piece AFOs are designed to support the patient's body weight during active non-neutral movement (e.g., rocking into the start and strike of the step), the aforementioned strapping embodiments do not. In fact, the underlying patient's condition/instability often prevents the foot from being used in this manner to support the patient's body weight. As a result, the straps need only support the foot and ankle rather than the patient's body weight.

As a related tangent, rigid one-piece designs often do not comfortably fit under clothes. The vast majority of AFOs are for “in-shoe” use and do not have a tread-they must be used with shoes, even when used at home. While there are some “soled” AFOs, these can only be worn without shoes and must be much more robust and bulkier to handle uneven terrain without the benefit of the shoe's cushioning and tread. More directly, some customers may prefer a less intrusive AFO that could be used with or without shoes. Existing AFOs do not adequately address this desire.

To these ends, one exemplary embodiment of the present disclosure leverages the tensioning techniques described above into a lighter-weight sock-like garment. Instead of using straps, the sock-like AFO supports the foot with tensioned cables running through fabric. In some variants, the fabric can be made with lighter-weight materials that more uniformly cradle and support the foot. The sock-like characteristics of the AFO allow for comfortable use both with and without shoes.

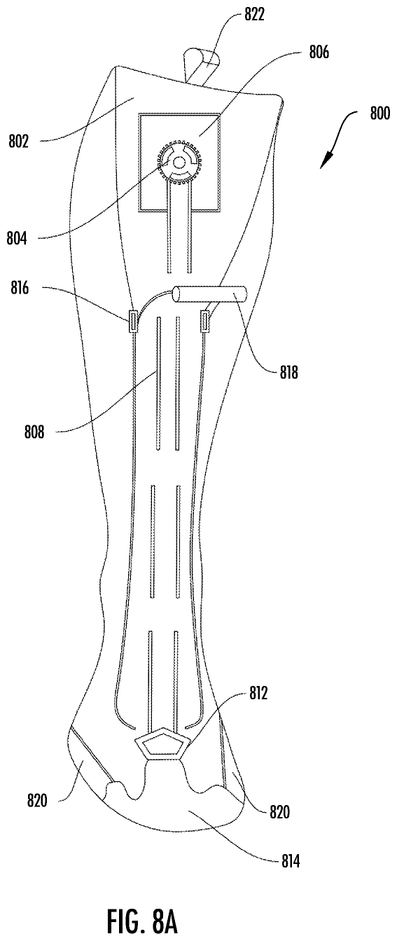

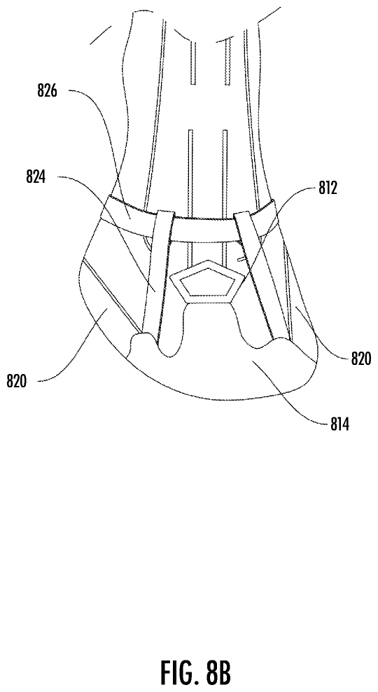

Referring now to FIG. 8A, one exemplary AFO 800 is shown and described. This AFO 800 has been integrated into a sock or garment. In some implementations, the AFO 800 includes a retention structure 802, a support structure for a rotary tensioning mechanism 804, support strap(s) (or cable(s)) 808, an attachment point 812, and supporting structure 814 disposed adjacent to the toes of a patient wearing the AFO 800. The underlying drop foot sock may, in some implementations, include low stretch in the horizontal direction in the region of the ankle and at the top of the calf (i.e., in the region of retention structure 802). The underlying drop foot sock may include a higher amount of stretch in the horizontal direction in one or more other regions to enable orthosis 800 to be put on, and taken off, easily, while also providing for improved comfort. Orthosis 800 may also include low stretch in the vertical direction to ensure proper stability of the dorsiflexion support strap/cable 808 and rotary tensioning mechanism 804.

As used herein, the term “fabric” refers to a textile that is woven, knit, or otherwise manufactured from fibers. Fibers are typically spun into yarn, and yarns are used to manufacture fabrics. Common examples of fibers include e.g., wool, silk, cotton, flax, nylon, polyester, acrylic, rayon, etc. Different weaves and knits may be used to affect directional tensile strengths; for example, certain knits can provide 2-way stretch in only one dimension. As noted above, different knits can be used to change the direction of tensile strength—e.g., directional stretch knits may be used to provide snug fit at anchor points (e.g., calf and ankle) and tensile strength.

Within the context of garments and fabrics, the term “stretch” refers to a fabric's ability to elastically stretch. Stretch fabrics may be classified as 2-way or 4-way; 2-way stretch fabrics only stretch in one dimension (e.g., width-wise or length-wise) whereas 4-way stretch fabrics can stretch in both dimensions. Generally, stretch fabrics are characterized by the difference between the maximally stretched fabric dimension versus its original dimension (at rest); thus, a fabric that stretches to twice its size has a stretch of 100%. High stretch fabrics often include synthetic and/or rubber materials e.g., Spandex, Lycra, etc. Knitted fibers generally have some amount of stretch—e.g., knitted wool, knitted cotton, etc. No stretch fabrics are usually cross woven fibers.

In one exemplary embodiment, the sock uses knit fabrics to achieve the desired stretch. Knit fabrics can offer both stretch, compression, and can be machine washed without issue. As previously noted, the AFO uses two different stretch weaves to implement both comfort and support. For example, 2-way stretch may be used to provide horizontal stretch for comfort, however vertical stretch may be limited to ensure that the supporting fabric can remain under sufficient tension. Similarly, 4-way stretch can be used to provide compression where desired (e.g., uniform compression of the calf muscle)

In particular, the low horizontal stretch ensures that the sock cuff and ankle both fit snugly and provide sufficient points of support for the AFO; the low vertical stretch ensures that the sock is stable. While similar effects can be created by using internal bracing structures and/or inserts, these may be unsuitable for use in a garment that is exposed to dirt and perspiration and must be washed often.

In the illustrated embodiment, support strap 808 is shown as an inelastic cable that is threaded within the underlying sock to enable, inter alia, the obviation of an ankle support strap. In some implementations, the inelastic cables are threaded in and out of the sock twice along the tibia region and threaded in again just above the ankle and out again adjacent the navicular bone of the foot. In some implementations, the support strap 808 (e.g., inelastic cable) may be threaded into the underside of sock (i.e., support strap 808 may be threaded so that portions thereof are disposed adjacent to, for example, the skin of a wearer of the AFO 800), while in other implementations the support strap 808 may be threaded inside of the sock such that the material of the sock is disposed between the skin of a wearer and the support strap 808 so as to mitigate irritation to the patient's skin by support strap 808 in some implementations. In some implementations, it may be desirable to thread support strap 808 entirely within the sock. Regardless of the implementation chosen, by virtue of the support strap 808 being threaded into (and out of) the underlying sock material (or within the underlying sock material), the support strap 808 may be placed proximate to the patient's foot/leg when the support strap 808 is placed under tension via, for example, rotary tensioning mechanism 804. In other implementations (not shown), the support strap may be disposed entirely (or almost entirely) within the sock (or underneath the sock) for the purpose of making orthosis 800 aesthetically pleasing. Preferably, the AFO 800 may be utilized with (or without) a standard set of shoes, boots, etc.

A connecting point 812 may comprise a plastic material having a curved routing cavity for receiving support strap 808. Additionally, in some implementations, a supporting structure 814 may be disposed adjacent to connecting point 812. The purpose of supporting structure 814 is to, inter alia, prevent the vertical stretch of the AFO 800 in this region when support strap 808 is placed under tension. In some implementations, both supporting structure 814 and connecting point 812 are attached to the underlying sock by being threaded thereon.

The retention structure 802 may be formed from the underlying sock material that enables the AFO 800 to remain secured above a patient's calf muscle without having to apply an additional tightening step. In other words, the retention structure 802 ensures that enough horizontal tension of the underlying sock material is placed around the top of a patient's calf muscle to enable the sock to stay in place when support strap 808 is tightened. Additionally, rotary tensioning dial 804 may be attached to a supporting fabric 806 which is sewn in the top region of orthosis 800. Accordingly, through use of supporting fabric 806, retention structure 802 and supporting structure 814, additional support may be provided to orthosis 800 to prevent the top of orthosis 800 from being pulled down over, for example, a patient's calf muscle when support strap 808 is placed under tension.

In one exemplary embodiment, the sock includes a variable-size cuff that snuggly fits around the user's calf when worn (“closed” or “zipped”) but can be opened to make donning/doffing more convenient (“opened” or “unzipped”). The illustrated embodiment uses a dual-zipper design that runs along the tibia of the patient; other implementations may use a single zipper (e.g., a zip up the back type design), or multiple zippers to provide different closure options.

Each zipper includes a slider mounted on two rows of metal or plastic teeth that are designed to interlock. The slider meshes or separates the teeth, depending on the direction of the slider's movement. Much like vertebra in a spine, the interlocking zipper teeth provide additional vertical support when fully closed. In other words, the interlocking teeth cannot be compressed or stretched, and create a flexible but sturdy assembly of fixed length. In the illustrated embodiment, the rotatory tensioning mechanism 804, support strap(s) 808, and attachment point 812 assembly lies between two zippers “spines”, thus most of the compressive force may be borne by the zipper teeth. Similar support structures may be used in other zipper configurations.

The AFO 800 may comprise dual zippers 816 running along the sides of the tibia region. Each zipper 816 may start from the top edge of the retention structure 802, run down along one side of the tibia region, and end near the hindfoot or the rear foot, but preferably near the midfoot. Each zipper 816 may have a slight inward curve before ending, wherein the slight inward curve prevents each zipper 816 from unzipping as easily. There may be a gap between the ends of the dual zippers 816, wherein the gap is wide enough to fit a rotary tensioning mechanism 804 if one were placed on foot region of the AFO 800. In another variation, the dual zippers 816 may be connected in the rear foot, midfoot, or hindfoot area.

The zipper 816 may be an invisible zipper wherein the zipper piece is hidden within the seam. In another variation, the zipper 816 may be an ordinary zipper 816. In another variation, a stretchable fabric may be sewn in between, or attached in between, edges of the retention structure 802 that have been separated by the zipper 816, wherein the stretchable fabric sits between a wearer's skin and the zipper 816 to improve comfort.

Commodity zippers closures are designed for able-bodied people; patients with low-dexterity in their hands may struggle to use a zipper pulls (much less two). To address this issue, the illustrated sock/garment AFO includes a zipper assist that connects two zippers pulls together. This provides multiple benefits, instead of grasping a zipper pull between the thumb and forefinger, the patient need only hook any of their fingers on the zipper assist to pull up. Additionally, instead of pulling up two zippers independently, the zipper assist allows both zippers to be zipped at the same time. While a particular zipper assist is depicted, other potential solutions may be substituted. For example, some zippers may use larger pulls or add lanyards.

The AFO 800 may further comprise a zipper assist 818, wherein the zipper assist 818 is comprised of a handle and strings, wherein each string has a first end and a second end, wherein each string is attached at the first end to one side of the handle and each string is attached at the second end to one of the pull tabs of the dual zippers 816 or the second end is incorporated into the dual zippers 816. The zipper assist 818 may aid in a single hand closure of the AFO 800; the zipper assist 818 zips up/down both sides simultaneously.

The AFO 800 may further comprise at least one pull tab 822 located at the top edge of the retention structure 802. The pull tabs may be positioned, in any combination, on the medial, lateral, front, and back sides of the AFO 800. In another variation, the pull tabs may be positioned in any other area on the AFO 800 than improves the ease of donning the AFO 800. While specific AFO variations have been contemplated, it would be readily appreciated by those in the field that the novel elements of dual zipper 816, the zipper assist 818, and at least one pull tab, individually, or in combination, may be readily adapted for use in other AFO variations.

As previously alluded to, patients with limited control over their toes and/or heel can have considerable difficult putting on socks. In large part, this is because the foot may get caught on the fabric as the sock is being pulled on. To address this issue, the sock/garment AFO includes a base member that the foot can push on. Other potential solutions may e.g., create a larger variable-size cuff, full-zip down, etc. In the illustrated embodiment, the base member allows the patient to put pressure on the foot during the donning process. This prevents catching since the material is stretched away from the foot. In some variants, the base member may also be used during wear to prevent “toe curling” and other similar conditions. In such cases, the base member may lift the toes or the lower half of the foot when put under tension by the attachment point 812 and support strap(s) (or cable(s)) 808. Depending on the nature of the foot condition, the sock may need a toe member, a midfoot member, a heel member, or possibly both.

The AFO 800 may further comprise a base member 820 located near the bottom front of the foot. The base member 820 may be incorporated into the AFO 800 or attached to the AFO through sewing, adhesives, or other common means. The shape and the material of the base member 820 makes it more rigid than the rest of the retention structure 802. The base member 820 may be generally thin, and aids in the process of donning the AFO 800, and acts as a stronger anchor point for a curved routing cavity. Notably, the resulting stronger curved routing cavity may help with donning, and also lift the toes and midfoot during use. Since the primary purpose of the sock-like AFO is to lift the toes to aid in walking without foot dropping, the in-use benefits to the stronger curved routing cavity may be significant. In another variation, the base member 820 encloses the entire sole of the foot.

In another variation, the base member 820 may cover the entire sole, be generally thicker, and be made from a semi-rigid material. Notably, modern shoe constructions may differentiate between insole, midsole, outsole, etc. Typically, an “insole” refers to a foot bed that provides cushioning and moisture wicking; in some cases, insoles may be removed/replaced from footwear. The midsole refers to the footwear's components that provide shock absorption, flex, support, and cushioning. The outsole provides wear resistance, traction, and tread. Most footwear designs treat the midsole and outsole as integral components that cannot be removed. As used herein, the term “sole” may refer to any combination of insole, midsole, and/or outsole components, either in whole or part.

In one exemplary embodiment, the base member may be formed from vulcanized rubber. Other semi-rigid materials suitable for sole composition include, without limitation: thermoplastic materials (PVC, TR, TPU, etc.), two component polyurethane materials (polyether PU, polyester PU, etc.), copolymers (such as rubber and EVA). The integration of such a base member 820 may allow a wearer to use the AFO 800 without wearing shoes while keeping his or her feet protected indoors.

In one specific implementation, the base member may additionally separate the toes to uniformly lift toes and alleviate toe curling afflictions. In one such implementation, the toes may be separated with rings, spacers, or other sections that serve to isolate each toe from its neighboring toe. Other implementations may include holes or depressions that toes can rest in. Still other solutions may be substituted with equal success.

While a specific AFO variation have been contemplated, it would be readily appreciated by those in the field that the novel element of a base member 820 may be readily adapted for use in other AFO variations.

Referring now to FIG. 8B, one exemplary AFO 800 is shown and described. The AFO 800 is similar to the AFO 800 shown in FIG. 8A but further comprises at least one anchor strap 824 and a connector strap 826. The anchor strap 824 has a first end and a second end, wherein the first end may be attached to, or integrated into, the supporting structure 814 and the second end may be attached to a D-ring or the second end forms a loop. The anchor strap 824 may be made of a tough fabric or another material with rigid or semi-rigid properties. The connector strap 826 has a first end and a second end, wherein the first end may be permanently attached to, or integrated into, the retention structure 802 or the base member 820 and the second end may be brought across over the midfoot, threaded through the D-rings or the loops of the anchor straps 824, and attached, using Velcro®, a button, or another suitable attachment method, to the retention structure 802 or the base member 820. The connector strap 826 may be made of a tough fabric or another material with rigid or semi-rigid properties. This arrangement of the anchor strap 824 and the connector strap 826 around the forefoot and midfoot region will allow the patient to easily put the AFO 800 on versus the flimsier sock material of the retention structure 802. During usage, the tensioning system may also dial up tension to lift the toes and/or midfoot area so the patient can walk better without dropping the toes.

While specific AFO variations have been contemplated, it would be readily appreciated by those in the field that the novel elements of at least one anchor strap 824 and the connector strap 826, individually, or in any combination, may be readily adapted for use in other AFO variations.

Some embodiments of the sock-like AFO may include enable embedded gait analysis capabilities. In one embodiment, the AFO may include e.g., the embedded programmable control module, battery pack, a communications module, pressure sensors, accelerometers, gyroscopes, and/or pedometers described above (see e.g., Ankle-Foot Orthoses with Integrated Gait Analysis). In some variants, the embedded gait analysis capabilities may be incorporated within the base member or sole components; alternatively, the sock-like AFO may include a separate pouch, pocket, patch, or similar compartment in the fabric for carrying the requisite electronics. Such implementations may be useful where the electronics are not machine wash safe and need to be removed, etc.

In some variants, the sock-like AFO may be able to provide embedded gait analysis capabilities, however the sock/garment may not have a foot plate or base member that is substantial enough to implement corrective adjustments. To address this, the patient may correct their gait using shoe inserts, pads, wedges, supports or other commodity orthotics. For example, a patient using a sock-like AFO may determine that they require corrective adjustment based on the vertical COM excursion data (or an patient-friendly nomenclature/indicia). In some such cases, they may take the gait analysis data to a clinician to prescribe (or a physical therapist to recommend) a suitable corrective orthotic. In other cases, an orthotics manufacturer may manufacture different orthotics, which are indexed according to the patient-friendly nomenclature/indicia. In this scenario, a patient could identify the suitable orthotic at their local pharmacy, monitor improvement, and adjust as needed without any further clinical supervision.

While the foregoing discussion is presented in the context of a light-weight sock/garment AFO, artisans of ordinary skill in the related arts, given the contents of the present disclosure, will readily appreciate that the sock-like AFO may be made more substantial with strapping and/or rigid inserts. Such implementations may be useful for hybrid products. As but one such example, a sock-like AFO may incorporate a complete rubberized sole and/or foot plate for outside usage. As another such example, the sock-like AFO may incorporate strut arm inserts such that a user can add additional structural support if needed. Notably, strut arm inserts may be used in medial, lateral, hammock strapping, anterior and/or posterior positioning, depending on desired usage (see e.g., Ankle-Foot Orthoses with Universal Strapping System, Ankle-Foot Orthoses with “Hammock” Support, Ankle-Foot Orthoses with Anterior/Posterior Support, etc.)

While the foregoing discussion describes a zipper closure that provides a variable-fit cuff, artisans of ordinary skill in the related arts, given the contents of the present disclosure, will readily appreciate that any variable-fit mechanism may be substituted with equal success. Other variable-fit mechanisms may rely on e.g., a hook and loop fastener (e.g., Velcro®), a clasp, a button, and/or any other suitable type of fastening mechanisms.

Additionally, the illustrated sock/garment AFO is depicted with a toe attachment point that is disposed in the center of the sock to provide dorsiflexion, however the techniques described herein may be broadly extended to other conditions/infirmities. For example, a sock/garment AFO could use an anchor point on the bottom of the sock, and run support strap(s) (or cable(s)) around the medial or lateral edge of the sock. In these configurations, tightening the rotary tensioning mechanism in these configurations would provide medial/lateral support. Similarly, a multi-tensioning sock/garment AFO could be used with both medial and lateral cabling to provide hammock-type support. Plantar flexion variants may use an anchor point on the top of the foot, which connects to cables run below the foot. Various other configurations may be substituted by artisans of ordinary skill in the related art, given the contents of the present disclosure.

Ankle-Foot Orthoses with Flexible Toe Plate

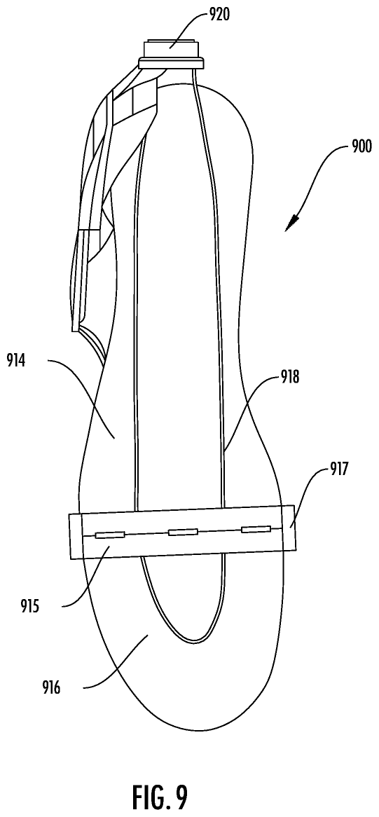

Referring now to FIG. 9, one exemplary AFO 900 is shown and described. This AFO 900 may comprise a foot plate 914, a flexible zone 915, a toe plate 916, cables 918, and a dial tensioning mechanism 920. The forward end of the foot plate 914 may be attached to, or integrated with, the back end of the flexible zone 915 and the forward end of the flexible zone 915 may attached to, or integrated with, the back end of the toe plate 916, wherein the foot plate 914, flexible zone 915, and toe plate 916 form the shape of a foot when combined.

The flexible zone 915 may be a spring-loaded hinge that permits the toe plate 916 to rotate upwards. The hinge may have a locking mechanism that prevents the angle between the bottom of the foot plate 914 and the bottom of the toe plate 916 from decreasing below 180 degrees. If a hinge is used for the flexible zone 915, the AFO may further comprise at least one rotational spring 917, wherein the rotational springs 917 exert a force on the toe plate 916 that rotates the toe plate 916 in the downwards direction.

In another variation, the flexible zone 915 may be a thin piece of semi-rigid material that slightly bends. The flexible zone 915 may be made of the same material as the foot plate 914 and the toe plate 916 but will be thinner than the foot plate 914 and the toe plate 916, allowing the thin piece of semi-rigid material to bend slightly. If a thin piece of semi-rigid material is used for the flexible zone 915, rotational springs 917 would not be required.

The dial tensioning mechanism 920 may be positioned on the back of a strut arm on the AFO 900. In another variation, the dial tensioning mechanism 920 may be positioned on the side of a strut arm. In yet other variations, dial tensioning mechanism 920 may be positioned on any part of the AFO that does not inhibit the functionality of the AFO 900.

Two cables 918 may each be attached at one end to the dial tensioning mechanism 920. The cables 918 may run, through the air, from the dial tensioning mechanism 920 to an opening of a cable channel near the back of the foot plate 914. The cables 918 may further run, through cable channels integrated into the foot plate 914, from the back of the of the foot plate 914, to the back of the flexible section 915. The cables 918 may further run, through cable channels integrated into the flexible section 915, from the back of the flexible section 915 to the back of the toe plate 916. The cables 918 may further run, through cable channels integrated into the toe plate 916, from the back of the toe plate 916, to near the front of the toe plate 916. The cables may be connected near the front of the toe plate 916. In another variation, the cables may run from the dial tensioning mechanism 920 to the foot plate 914 through cable channels integrated into the strut arms.

The dial tensioning system allows the wearer of the AFO 900 to tighten the cables 918 with one hand. When the cables 918 are tightened, the toe plate 916 rotates upwards, lifting the toe plate 916 to aid a drop foot patient when his or her foot is in swing phase to give toe clearance.

While specific AFO variations have been contemplated, it would be readily appreciated by those in the field that the novel elements of a foot plate 914, a flexible zone 915, a toe plate 916, cables 918, and a dial tensioning mechanism 920, individually, or in any combination, may be readily adapted for use in other AFO variations.

### Material Considerations for Ankle-Foot Orthoses Subcomponents

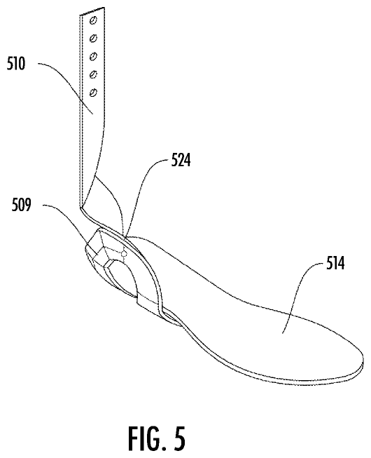

Referring now to FIG. 5, one exemplary AFO 500 comprising a bottom strut dual arm 509, a top strut arm 510, a foot plate 514, and an angular adjustment gear 524 is shown and described in detail. The bottom strut dual arm 509 and the top strut arm 510 allow the AFO to be reconfigured between posterior or anterior configurations.

Each strut arm and the foot plate 514 may be made of different materials including, but not limited to, carbon fiber, fiber glass, and thermoplastics. For example, the foot plate may be made of carbon fiber and the strut arms may be made of fiber glass. In this case, the fiber glass may provide more flexibility and energy return than the carbon fiber, whereas the carbon fiber may be lighter and easier to fit to the foot. Such material diversity can be used to offer strategic flexibility and stiffness to enhance performance and patient comfort based on patients' needs. The angular adjustment gear 524 may provide additional adjustment of stiffness.

While a specific AFO variation has been contemplated, it would be readily appreciated by those in the field that the use of different materials for different AFO 500 parts may be readily adapted for use in other AFO variations.

Where certain elements of these implementations can be partially or fully implemented using known components, only those portions of such known components that are necessary for an understanding of the present disclosure are described, and detailed descriptions of other portions of such known components are omitted so as not to obscure the disclosure.

In the present specification, an implementation showing a singular component should not be considered limiting; rather, the disclosure is intended to encompass other implementations including a plurality of the same component, and vice versa, unless explicitly stated otherwise herein.

Further, the present disclosure encompasses present and future known equivalents to the components referred to herein by way of illustration.

It will be recognized that while certain aspects of the technology are described in terms of a specific sequence of steps of a method, these descriptions are only illustrative of the broader methods of the disclosure and may be modified as required by the particular application. Certain steps may be rendered unnecessary or optional under certain circumstances. Additionally, certain steps or functionality may be added to the disclosed implementations, or the order of performance of two or more steps permuted. All such variations are considered to be encompassed within the disclosure disclosed and claimed herein.

While the above detailed description has shown, described, and pointed out novel features of the disclosure as applied to various implementations, it will be understood that various omissions, substitutions, and changes in the form and details of the device or process illustrated may be made by those skilled in the art without departing from the disclosure. The foregoing description is of the best mode presently contemplated of carrying out the principles of the disclosure. This description is in no way meant to be limiting, but rather should be taken as illustrative of the general principles of the technology. The scope of the disclosure should be determined with reference to the claims.

It will be apparent to those skilled in the art that various modifications and variations can be made in the disclosed embodiments of the disclosed device and associated methods without departing from the spirit or scope of the disclosure. Thus, it is intended that the present disclosure covers the modifications and variations of the embodiments disclosed above provided that the modifications and variations come within the scope of any claims and their equivalents.

What is claimed is:

1. An ankle-foot orthosis, comprising:
a first fitting configured to fit adjacent to a knee of a user;
a first strut arm attached to the first fitting;
a foot plate configured to support a foot of the user;
a second strut arm attached to the foot plate;
where the first strut arm and the second strut arm are configured to mate according to one of a plurality of angular positions; and
a first strap anchored to the foot plate and configured to attach to the first fitting.

2. The ankle-foot orthosis of claim 1, where the first strap is configured to wrap around a leg of the user to provide a first amount of tension while the foot is in a relaxed position and provide a second amount of tension during dorsiflexion or plantar flexion.

3. The ankle-foot orthosis of claim 1, where the first strut arm and the second strut arm are configured to mate according to one of the plurality of angular positions.

4. The ankle-foot orthosis of claim 3, where the first strut arm comprises a first set of gear teeth, the second strut arm comprises a second set of gear teeth, and the first set of gear teeth fit into empty spaces of the second set of gear teeth.

5. The ankle-foot orthosis of claim 1, where the first strap is anchored to a slot of the foot plate.

6. The ankle-foot orthosis of claim 1, where the foot plate comprises a hammock to support both a medial and a lateral side of the foot.

7. The ankle-foot orthosis of claim 1, where the second strut arm is characterized by a horseshoe shape configured to support a medial malleolus or a lateral malleolus of the foot.

8. An ankle-foot orthosis, comprising:
a support structure to fit adjacent to a knee of a user;
a foot plate configured to support a foot of the user;
where the foot plate further comprises an accelerometer, a gyroscope, and a plurality of pressure sensors;
a first strap anchored to the foot plate and configured to attach to the support structure;
a processor; and
a non-transitory computer-readable medium comprising one or more instructions that, when executed by the processor, cause the processor to:
measure step data;
measure pressure data at a plurality of points along the foot for at least the step data; and
perform a gait analysis based on the step data and the pressure data.

9. The ankle-foot orthosis of claim 8, where the first strap is configured to wrap around a leg of the user to provide a first amount of tension while the foot is in a relaxed position and provide a second amount of tension during dorsiflexion or plantar flexion.

10. The ankle-foot orthosis of claim 8, where the gait analysis is performed over a period of time.

11. The ankle-foot orthosis of claim 8, where the gait analysis is performed over a number of steps.

12. The ankle-foot orthosis of claim 8, where the support structure further comprises an accelerometer and a gyroscope; and
where the one or more instructions, when executed by the processor, cause the processor to perform a kinematic analysis of the knee relative to the foot.

13. The ankle-foot orthosis of claim 8, further comprising a heart rate monitor or a blood oxygen sensor; and
where the one or more instructions, when executed by the processor, cause the processor to perform a metabolic analysis.

14. The ankle-foot orthosis of claim 8, further comprising a time reference;
where the one or more instructions, when executed by the processor, cause the processor to synchronize the time reference relative to at least one other ankle-foot orthosis; and
record the step data and the pressure data according to the time reference.

15. An ankle-foot orthosis, comprising:
a retention structure configured to fit adjacent to a knee of a user;
a fabric sock configured to support a foot of the user;
a first strap anchored to the fabric sock and attached to a rotary tensioning system; and
at least a first zipper closure configured to connect the rotary tensioning system to the retention structure.

16. The ankle-foot orthosis of claim 15, further comprising a second zipper closure configured to connect the rotary tensioning system to the retention structure; and
where the first zipper closure and the second zipper closure are configured to run along a tibia of the user.

17. The ankle-foot orthosis of claim 16, further comprising a zipper assist connected to the first zipper closure and the second zipper closure.

18. The ankle-foot orthosis of claim 15, where the fabric sock comprises a rigid base member proximate to a front of the foot.

19. The ankle-foot orthosis of claim 15, where the fabric sock comprises a rigid base member along an entire sole of the foot.

20. The ankle-foot orthosis of claim 15, where the fabric sock can be removed from the ankle-foot orthosis for washing.
